(19) United States
(12) Patent Application Publication
(10) Pub. No.: US 2010/0305174 A1
(43) Pub. Date: Dec. 2, 2010

(54) Novel Heterocyclic Derivatives

(75) Inventors: Surendrakumar Satyanarayan Pandey, Chennai (IN); Gadam Om Reddy, Chennai (IN); Shiv Kumar Agarwal, New Delhi (IN); Gajendra Singh, Chennai (IN); Santhanagopalan Chithra, Chennai (IN); Debendranath Dey, Union City, CA (US); Abhijeet Nag, Union City, CA (US); Kadnur Venkatachalapathi Sanjay, Chennai (IN)

Correspondence Address: SUGHRUE MION, PLLC, 2100 PENNSYLVANIA AVENUE, N.W., SUITE 800, WASHINGTON, DC, 20037, US

(73) Assignee: ORCHID RESEARCH LABORATORIES LIMITED, Chennai (IN)

(21) Appl. No.: 11/918,235

(22) PCT Filed: Apr. 12, 2006
(86) PCT No.: PCT/IB2006/000854

§ 371 Date: Jun. 23, 2010

(30) Foreign Application Priority Data
Apr. 13, 2005 (IN) 421/CHE/2005

(51) Int. Cl. A61K 31/427; C07D 417/12; C07D 413/12; A61K 31/422; A61P 3/10; A61P 3/06; A61P 3/04; A61P 37/00; A61P 29/00; A61P 35/00

(52) U.S. Cl. 514/369; 548/183; 548/229; 514/376

(57) ABSTRACT

The present invention relates to novel heterocyclic derivatives of formula (I) and their pharmaceutically acceptable salts, their derivatives, their analogs, their tautomeric forms, their stereoisomers, their polymorphs, their hydrates, their solvates, their pharmaceutically acceptable salts, their pharmaceutical compositions, and their prodrugs thereof. The present invention more particularly provides novel compounds of the general formula (1).

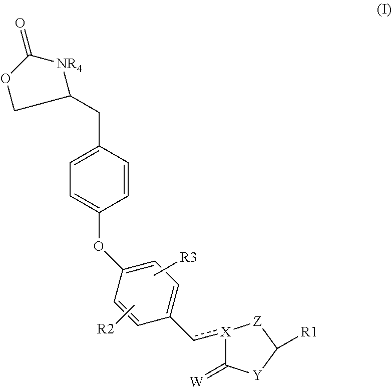

### FIELD OF THE INVENTION

The present invention relates to novel heterocyclic derivatives of formula (I) and their pharmaceutically acceptable salts, their derivatives, their analogs, their tautomeric forms, their stereoisomers, their polymorphs, their hydrates, their solvates, their pharmaceutically acceptable salts, their pharmaceutical compositions, and their prodrugs thereof. The present invention more particularly provides novel compounds of the general formula (I).

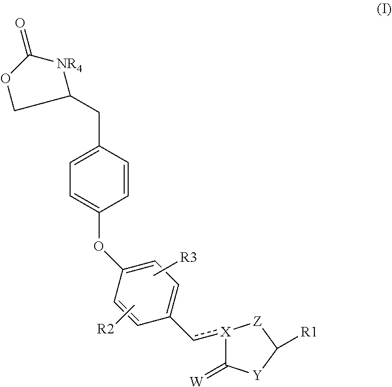

The present invention also relates to a process for the preparation of the above said novel compounds and their pharmaceutically acceptable salts. The compounds of the present invention are effective in lowering blood glucose, serum insulin, free fatty acids, cholesterol and triglyceride levels and are useful in the treatment and/or prophylaxis of type II diabetes. The compounds of the present invention are effective in treatment of obesity, inflammation, autoimmune diseases such as multiple sclerosis and rheumatoid arthritis. Surprisingly, these compounds increase the leptin level and have no liver toxicity.

Furthermore, the compounds of the present invention are useful for the treatment of disorders associated with insulin resistance, such as polycystic ovary syndrome, as well as hyperlipidemia, coronary artery disease and peripheral vascular disease, and for the treatment of inflammation and immunological diseases, particularly those mediated by cytokines such as TNF-α, IL-1, IL-6, and IL-1β.

### BACKGROUND OF THE INVENTION

The causes of type I and II diabetes are not yet clear, although both genetics and environment seem to be the factors. Type I is an autonomic immune disease and patient must take insulin to survive. Type II diabetes is more common form is a metabolic disorder resulting from the body's inability to make a sufficient amount of insulin or to properly use the insulin that is produced. Insulin secretion and insulin resistance are considered the major defects, however, the precise genetic factors involved in the mechanism remain unknown.

Patients with diabetes usually have one or more of the following defects:

Less production of insulin by the pancreas;

Over secretion of glucose by the liver;

Independent of the glucose uptake by the skeletal muscles;

Defects in glucose transporters, desensitization of insulin receptors; and

Defects in the metabolic breakdown of polysaccharides.

Other than the parenteral or subcutaneous administration of insulin, there are about four classes of oral hypoglycemic agents used i.e sulfonylurea, biguanides, alpha glucosidase inhibitors and thiazolidinediones.

Each of the current agents available for use in treatment of diabetes has certain disadvantages. Accordingly, there is a continuing interest in the identification and development of new agents, which can be orally administered, for use in the treatment of diabetes.

The thiazolidinedione class listed above has gained more widespread use in recent years for treatment of type II diabetes, exhibiting particular usefulness as insulin sensitizers to combat “insulin resistance”, a condition in which the patient becomes less responsive to the effects of insulin. There is a continuing need for nontoxic, more widely effective insulin sensitizers. In our present invention we explore new compounds having antidiabetic activity, we propose to synthesis new compounds containing rhodanine, rhodanine-3-acetic acid, thiazolidinone, oxindole, and oxazolidinone system and study them by taking thiazolidinone as comparator.

Recent advances in scientific understanding of the mediators involved in acute and chronic inflammatory diseases and cancer have led to new strategies in the search for effective therapeutics. Traditional approaches include direct target intervention such as the use of specific antibodies, receptor antagonists, or enzyme inhibitors. Recent breakthroughs in the elucidation of regulatory mechanisms involved in the transcription and translation of a variety of mediators have led to increased interest in therapeutic approaches directed at the level of gene transcription.

As indicated above, the present invention is also concerned with treatment of immunological diseases or inflammation, notably such diseases as are mediated by cytokines or cyclooxygenase. The principal elements of the immune system are macrophages or antigen-presenting cells, T cells and B cells. The role of other immune cells such as NK cells, basophils, mast cells and dendritic cells are known, but their role in primary immunologic disorders is uncertain. Macrophages are important mediators of both inflammation and providing the necessary “help” for T cell stimulation and proliferation. Most importantly macrophages make IL 1, IL 12 and TNF-α all of which are potent pro-inflammatory molecules and also provide help for T cells. Many factors activate macrophages, including bacterial products, superantigens and interferon gamma (IFNγ). It is believed that phosphotyrosine kinases (PTKs) and other undefined cellular kinases are involved in the activation process.

Few Prior Art Reference which Disclose the Closest Compounds are Given Here:

i) International publication No. WO 01/02377 discloses compounds of formula (IIa) as telomerase inhibitors

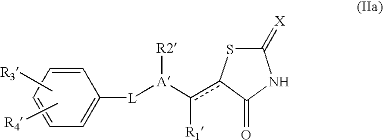

wherein R′₁ and R′₂ represents hydrogen, alkyl etc., X represents oxygen or sulfur; [symbol] is a single or double bond; L represents oxygen, nitrogen, sulfur; R′₃ represents hydrogen, alkyl, aryl etc., R′₄ represents hydrogen, alkyl, aryl etc., A′ represents aryl.

An example of these compounds is shown in formula (IIb)

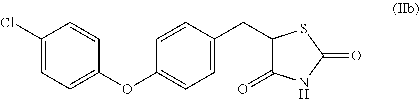

ii) EP 1148054 discloses compounds of formula (IIc)

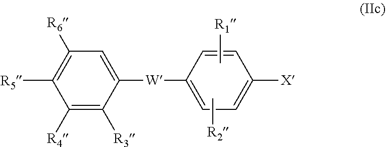

wherein R₁″, R₂″, R₃″, R₅″, R₆″, represent hydrogen, alkyl etc., X′ represents methylene thiazolidin-2,4-dione, methylene oxazolidin-2,4-dione etc., W′ represents oxygen, sulfur; R₄″ represents hydrogen, alkyl substituted with zero to three substituents etc.

An example of these compounds is shown in formula (IId)

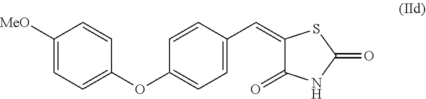

iii) U.S. Pat. No. 6,331,633 discloses compounds of formula (IIe)

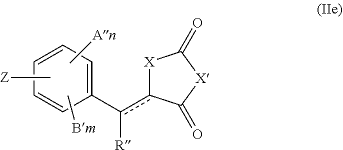

wherein Z is

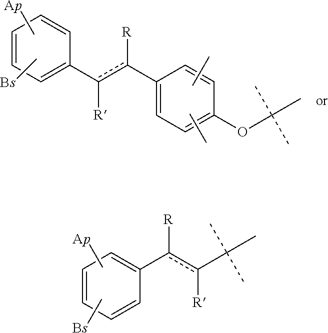

wherein n, m, q and r are independently integers from zero to 4; p and s are independently integers from zero to 5; a, b and c are double bonds which may be present or absent; R, R′ and R″ are independently H, C₁-C₂₀ linear or branched alkyl; C₂-C₂₀ linear or branched alkenyl, —CO₂H, —CO₂R′″, —NH₂, —NHR′″, —NR₂′″, —OH, —OR′41 , halo, substituted C₁-C₂₀ linear or branched alkyl or substituted C₂-C₂₀ linear or branched alkenyl, wherein R′″ is C₁-C₂₀ linear or branched alkyl or linear or branched alkenyl; A, A′ and A″ are independently H, C₁-C₂₀acylamino; C₁-C₂₀acyloxy; C₁-C₂₀alkanoyl; C₁-C₂₀alkoxycarbonyl; C₁-C₂₀alkoxy; C₁-C₂₀alkylamino; C₁-C₂₀alkylcarboxylamino; carboxyl; cyano; halo; hydroxy; B, B′ and B″ are independently H; C₁-C₂₀acylamino; C₁-C₂₀ acyloxy; C₁-C₂₀ alkanoyl; etc., X, X′ are independently —NH, —NR′″, O or S.

An example of these compounds is shown in formula (IIf)

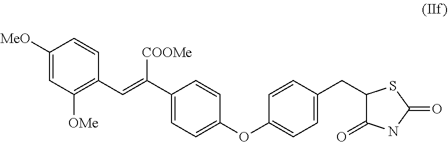

iv) Tetrahedron asymmetry 14 (2003) 2619-2623 disclose the four step synthesis of enantiopure (S)-4-(4-hydroxybenzyl)-oxazolidin-2-one (S-1) from N-Boc-L-tyrosine and discloses the intermediate of formula (S)-1.

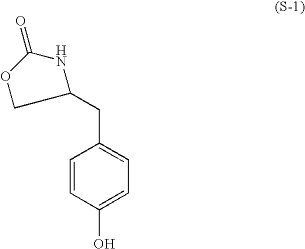

### OBJECTIVE OF THE INVENTION

With an objective to develop novel compounds for lowering blood glucose, free fatty acids, cholesterol and triglyceride levels in type II diabetes and to treat autoimmune diseases such as multiple sclerosis and rheumatoid arthritis, we focused our research to develop new compounds effective in the treatment of the above mentioned diseases. Efforts in this direction have led to compounds having general formula (I).

The main objective of the present invention is therefore, to provide novel heterocyclic derivatives and their pharmaceutically acceptable salts.

Another objective of the present invention is to provide novel heterocyclic derivatives and their pharmaceutically acceptable salts having enhanced activities, without toxic effect or with reduced toxic effect.

Yet another objective of the present invention is to provide a process for the preparation of novel heterocyclic derivatives of formula (I), their pharmaceutically acceptable salts.

### SUMMARY OF THE INVENTION

The present invention, relates to novel heterocyclic derivatives of formula (I)

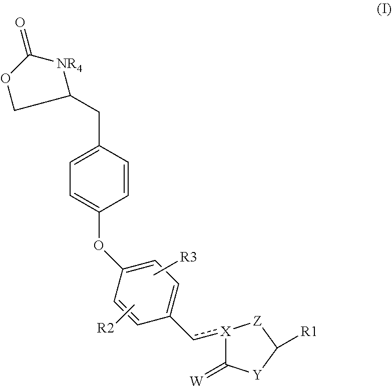

their pharmaceutically acceptable salts, their derivatives, their analogs, their tautomeric forms, their stereoisomers, their polymorphs, their hydrates, their solvates, their pharmaceutically acceptable salts, their pharmaceutical compositions, and their prodrugs thereof; wherein [symbol] represents optional bond; W represents O or S; X represents C, CH or N; Y represents NR₅, S or O, wherein R₅ represents hydrogen, substituted or unsubstituted alkyl, alkenyl, —CH₂COOR, or aryl, or counter ion; wherein R represents H or alkyl group; Z represents CR₆ or S; R₁ represents ═O, ═S or together with R₆ forms fused 5 or 6 membered aromatic or heteroaromatic ring system containing carbon atoms or 1 or 2 heteroatoms selected from O, S or N; R₂, R₃, may be same or different and independently represent hydrogen, halogen, hydroxy, nitro, cyano, formyl, amino, alkyl, haloakyl, alkoxy group; R₄ may be same or different and independently represent H, COR₇, substituted or unsubstituted groups selected from alkyl, alkenyl, aryl,aryloxy,alkoxy, heteroaryl or heterocyclyl; where R₇ represents H, substituted or unsubstituted groups selected from alkyl, alkenyl, aryl, aryloxy, alkoxy or aralkoxy.

### DETAILED DESCRIPTION OF THE INVENTION:

Suitable groups represented by R₁ are selected from ═O, ═S; or together with R₆ forms fused 5 or 6 membered aromatic or heteroaromatic ring system containing carbon atoms or 1 or 2 hetereoatoms selected form O, S or N such as phenyl, naphthyl, furyl, pyrrolyl, pyridyl and the like.

Suitable groups represented by R₂, R₃, are selected from hydrogen, halogen such as fluorine, chlorine, bromine or iodine; hydroxy, nitro, cyano, formyl, amino, substituted or unsubstituted linear or branched, (C₁-C₄) alkyl group such as methyl, ethyl, propyl, isopropyl, n-butyl, isobutyl, t-butyl, and the like; haloalkyl groups selected from alkyl group substituted by one, two, three or four halogen atoms such as chloromethyl, chloroethyl, trifluoromethyl, trifluoroethyl, dichloromethyl, dichloroethyl and the like. Substituted or unsubstituted (C₁-C₄) alkoxy group such as methoxy, ethoxy, propoxy, butoxy and the like.

Suitable groups represented by R₄ may be same or different and independently represent H, alkyl, alkenyl, substituted or unsubstituted groups selected from (C₁-C₄) alkyl group such as methyl, ethyl, propyl, isopropyl, n-butyl, isobutyl, t-butyl and the like; Substituted or unsubstituted linear or branched C₂-C₇ alkenyl such as ethenyl, propenyl, butenyl and the like; aryl group such as phenyl, naphthyl and the like, the aryl group may be substituted; aryloxy, substituted or unsubstituted linear or branched C₂-C₂₀ alkoxy such as methoxy, ethoxy, propoxy, n-butoxy, isobutoxy, t-butoxy and the like; heteroaryl group such as pyridyl, thienyl, furyl, pyrrolyl, oxazolyl, thiazolyl, imidazolyl, oxadiazolyl, triazolyl, tetrazolyl, pyrimidinyl, pyrazinyl, indolyl, indolinyl, benzothiazolyl, and the like, which may be substituted; heterocyclyl group such as pyrrolidinyl, thiazolidinyl, oxazolidinyl, morpholinyl, thiomorpholinyl, piperidinyl, piperazinyl, and the like, which may be substituted, COR₇; where R₇ represents H; substituted or unsubstituted groups selected from (C₁-C₄) alkyl group such as methyl, ethyl, propyl, isopropyl, n-butyl, isobutyl, t-butyl and the like; Substituted or unsubstituted linear or branched C₂-C₇ alkenyl such as ethenyl, propenyl, butenyl and the like; aryl group such as phenyl, naphthyl and the like, the aryl group may be substituted; aryloxy, substituted or unsubstituted liner or branched C₂-C₂₀ alkoxy such as methoxy, ethoxy, propoxy, n-butoxy, isobutoxy, t-butoxy and the like;

Pharmaceutically acceptable salts forming part of this invention include base addition salts such as alkali metal salts like Li, Na, and K salts, alkaline earth metal salts like Ca and Mg salts, salts of organic bases such as lysine, arginine, guanidine, diethanolamine, choline and the like, ammonium or substituted ammonium salts. Salts may include acid addition salts which are sulphates, nitrates, phosphates, perchlorates, borates, hydrohalides, acetates, tartrates, maleates, citrates, succinates, palmoates, methanesulphonates, benzoates, salicylates, hydroxynaphthoates, benzenesulfonates, ascorbates, glycerophosphates, ketoglutarates and the like. Pharmaceutically acceptable solvates may be hydrates or comprising other solvents of crystallization such as alcohols.

The pharmaceutical composition may be in the forms normally employed, such as tablets, capsules, powders, syrups, solutions, suspensions and the like, may contain flavourants, sweeteners etc. in suitable solid or liquid carriers or diluents, or in suitable sterile media to form injectable solutions or suspensions. Such compositions typically contain from 1 to 25%, preferably 1 to 15% by weight of active compound, the remainder of the composition being pharmaceutically acceptable carriers, diluents, excipients or solvents.

Suitable pharmaceutically acceptable carriers include solid fillers or diluents and sterile aqueous or organic solutions. The active compound will be present in such pharmaceutical compositions in the amounts sufficient to provide the desired dosage in the range as described above. Thus, for oral administration, the compounds can be combined with a suitable solid or liquid carrier or diluent to form capsules, tablets, powders, syrups, solutions, suspensions and the like. The pharmaceutical compositions, may, if desired, contain additional components such as flavourants, sweeteners, excipients and the like. For parenteral administration, the compounds can be combined with sterile aqueous or organic media to form injectable solutions or suspensions. For example, solutions in sesame or peanut oil, aqueous propylene glycol and the like can be used, as well as aqueous solutions of water-soluble pharmaceutically-acceptable acid addition salts or alkali or alkaline earth metal salts of the compounds. The injectable solutions prepared in this manner can then be, administered intravenously, intraperitoneally, subcutaneously, or intramuscularly, with intramuscular administration being preferred in humans.

The pharmaceutical composition of the present invention are effective in lowering blood glucose, serum insulin and triglyceride levels in animal models of types II diabetes. The pharmaceutical compositions of the present invention are also effective in the treatment of obesity, inflammation, and autoimmune diseases. Furthermore, pharmaceutical composition of the present invention are useful for the treatment of disorders associated with insulin resistance, such as polycystic ovary syndrome, as well as hyperlipidemia, coronary artery disease and peripheral vascular disease, and for the treatment of inflammation and immunological diseases, particularly those mediated by cytokines such as TNF-α, IL-1β, and IL-6.

The protecting group used in the invention are conventional protecting groups such as t-butoxycarbonyl(t-Boc), trityl, trifluoroacetyl, benzyloxy, benzyloxy carbonyl (Cbz) and the like. Deprotection can be done by conventional methods.

Particularly Useful Compounds According to the Invention Include:
1) 5-(4-{4-[(3-methyl-2-oxo-1,3-oxazolidin-4-yl)methyl]phenoxy}benzylidene)-1,3-thiazolidine-2,4-dione;
2) 5-(4-{4-[(2-oxo-1,3-oxazolidin-4-yl)methyl]phenoxy}benzylidene)-1,3-thiazolidine-2,4-dione;
3) 5-(4-{4-[(3-ethyl-2-oxo-1,3-oxazolidin-4-yl)methyl]phenoxy}benzylidene)-1,3-thiazolidine-2,4-dione;
4) 5-(4-{4-[(3-methyl-2-oxo-1,3-oxazolidin-4-yl)methyl]phenoxybenzylidene)-1,3-dihydro-2H-indol-2-one;
5) 5-(4-{4-[(3-ethyl-2-oxo-1,3-oxazolidin-4-yl)methyl]phenoxybenzylidene)-1,3-dihydro-2H-indol-2-one;
6) 5-(3-fluoro-4-{4-[(3-methyl-2-oxo-1,3-oxazolidin-4-yl)methyl]phenoxy}benzylidene)-1,3-thiazolidine-2,4-dione;
7) 5-(3-fluoro-4-{4-[(3-methyl-2-oxo-1,3-oxazolidin-4-yl)methyl]phenoxybenzylidene)-1,3-dihydro-2H-indol-2-one;
8) 5-(3-chloro-4-{4-[(3-methyl-2-oxo-1,3-oxazolidin-4-yl)methyl]phenoxy}benzylidene)-1,3-thiazolidine-2,4-dione;
9) 5-(3-chloro-4-{4-[(3-methyl-2-oxo-1,3-oxazolidin-4-yl)methyl]phenoxybenzylidene)-1,3-dihydro-2H-indol-2-one;
10) 5-(3-chloro-4-{4-[(3-ethyl-2-oxo-1,3-oxazolidin-4-yl)methyl]phenoxybenzylidene)-1,3-dihydro-2H-indol-2-one;
11) 5-(3-methoxy-4-{4-[(3-methyl-2-oxo-1,3-oxazolidin-4-yl)methyl]phenoxybenzylidene)-1,3-dihydro-2H-indol-2-one;
12) 5-(3-fluoro-4-{4-[(3-ethyl-2-oxo-1,3-oxazolidin-4-yl)methyl]phenoxybenzylidene)-1,3-dihydro-2H-indol-2-one;
13) 3-(4-{3-methoxy-4-[(3-ethyl-2-oxo-1,3-oxazolidin-5-yl)methyl]phenoxy}benzylidene)-1,3-dihydro-2H-indol-2-one;
14) 3-(4-{3-triflouromethyl-4-[(3-methyl-2-oxo-1,3-oxazolidin-5-yl)methyl]phenoxy}benzylidene)-1,3-dihydro-2H-indol-2-one;
15) 3-(4-{3-triflouromethyl-4-[(3-ethyl-2-oxo-1,3-oxazolidin-5-yl)methyl]phenoxy}benzylidene)-1,3-dihydro-2H-indol-2-one;
16) 5-(2-chloro-4-{4-[(3-methyl-2-oxo-1,3-oxazolidin-4-yl)methyl]phenoxybenzylidene)-1,3-dihydro-2H-indol-2-one;
17) 3-(4-{2-chloro-4-[(3-ethyl-2-oxo-1,3-oxazolidin-5-yl)methyl]phenoxy}benzylidene)-1,3-dihydro-2H-indol-2-one
18) 5-(3-trifluoromethyl-4-{4-[(3-methyl-2-oxo-1,3-oxazolidin-4-yl)methyl]phenoxy}benzylidene)-1,3-thiazolidine-2,4-dione;
19) 5-(3-methoxy-4-{4-[(3-methyl-2-oxo-1,3-oxazolidin-4-yl)methyl]phenoxy}benzylidene)-1,3-thiazolidine-2,4-dione;
20) 5-(2-chloro-4-{4-[(3-methyl-2-oxo-1,3-oxazolidin-4-yl)methyl]phenoxy}benzylidene)-1,3-thiazolidine-2,4-dione;
21) 5-(3-chloro-4-{4-[(3-ethyl-2-oxo-1,3-oxazolidin-4-yl)methyl]phenoxy}benzylidene)-1,3-thiazolidine-2,4-dione;
22) 5-(3-fluoro-4-(4-[(3-ethyl-2-oxo-1,3-oxazolidin-4-yl)methyl]phenoxy}benzylidene)-1,3-thiazolidine-2,4-dione;
23) 5-(3-methoxy-4-{4-[(3-ethyl-2-oxo-1,3-oxazolidin-4-yl)methyl]phenoxy}benzylidene)-1,3-thiazolidine-2,4-dione;
24) 5-(3-triflouromethyl-4-{4-[(3-ethyl-2-oxo-1,3-oxazolidin-4-yl)methyl]phenoxy}benzylidene)-1,3-thiazolidine-2,4-dione;
25) 5-(2-chloro-4-{4-[(3-ethyl-2-oxo-1,3-oxazolidin-4-yl)methyl]phenoxy}benzylidene)-1,3-thiazolidine-2,4-dione;
26) 5-(2-fluoro-4-{4-[(3-ethyl-2-oxo-1,3-oxazolidin-4-yl)methyl]phenoxy}benzylidene)-1,3-thiazolidine-2,4-dione;
27) 5-(2-fluoro-4-{4-[(3-'methyl-2-oxo-1,3-oxazolidin-4-yl)methyl]phenoxy}benzylidene)-1,3-thiazolidine-2,4-dione;
28) 3-(4-{3-fluoro-4-[(2-oxo-1,3-oxazolidin-5-yl)methyl]phenoxy}benzylidene)-1,3-dihydro-2H-indol-2-one;
29) 5-(3-chloro-4-{4-[(2-oxo-1,3-oxazolidin-4-yl)methyl]phenoxy}benzylidene)-1,3-thiazolidine-2,4-dione;
30) 3-(4-{3-chloro-4-[(2-oxo-1,3-oxazolidin-5-yl)methyl]phenoxy}benzylidene)-1,3-dihydro-2H-indol-2-one;
31) 5-(3-fluoro-4-{4-[(2-oxo-1,3-oxazolidin-4-yl)methyl]phenoxy}benzylidene)-1,3-thiazolidine-2,4-dione;
32) [(4-oxo-5-(4-{4-[(3-methyl-2-oxo-1,3-oxazolidin-4-yl)methyl]phenoxy}benzylidene)-2-thioxo-1,3-thiazolidin-3-yl]acetic acid;
33) 4-(4-{4-[(4-oxo-2-thioxo-1,3-thiazolidin-5-ylidene)methyl]phenoxy}benzyl)-1,3-oxazolidin-2-one;
34) 3-methyl-4-(4-{4-[(4-oxo-2-thioxo-1,3-thiazolidin-5-ylidene)methyl]phenoxy}benzyl)-1,3-oxazolidin-2-one;
35) 3-ethyl-4-(4-{4-[(4-oxo-2-thioxo-1,3-thiazolidin-5-ylidene)methyl]phenoxy}benzyl)-1,3-oxazolidin-2-one;
36) [(4-oxo-5-(4-{4-[(2-oxo-1,3-oxazolidin-4-yl)methyl]phenoxy}benzylidene)-2-thioxo-1,3-thiazolidin-3-yl]acetic acid;
37) [(4-oxo-5-(4-{4-[(3-ethyl-2-oxo-1,3-oxazolidin-4-yl)methyl]phenoxy}benzylidene)-2-thioxo-1,3-thiazolidin-3-yl]acetic acid;
38) 4-(4-{2-fluoro-4-[(4-oxo-2-thioxo-1,3-thiazolidin-5-ylidene)methyl]phenoxy}benzyl)-3-methyl-1,3-oxazolidin-2-one;
39) [(4-oxo-5-(3-fluoro-4-{4-[(3-methyl-2-oxo-1,3-oxazolidin-4-yl)methyl]phenoxy}benzylidene)-2-thioxo-1,3-thiazolidin-3-yl]acetic acid;
40) 4-(4-{2-chloro-4-[(4-oxo-2-thioxo-1,3-thiazolidin-5-ylidene)methyl]phenoxy}benzyl)-3-methyl-1,3-oxazolidin-2-one;
41) [4-oxo-5-(3-chloro-4-{4-[(3-methyl-2-oxo-1,3-oxazolidin-4-yl)methyl]phenoxy}benzylidene)-2-thioxo-1,3-thiazolidin-3-yl]acetic acid;
42) 4-(4-{2-fluoro-4-[(4-oxo-2-thioxo-1,3-thiazolidin-5-yl)methyl]phenoxy}benzylidene)-3-ethyl-1,3-oxazolidin-2-one;
43) 4-(4-{2-methoxy-4-[(4-oxo-2-thioxo-1,3-thiazolidin-5-yl)methyl]phenoxy}benzylidene)-3-ethyl-1,3-oxazolidin-2-one;
44) 4-(4-{2-chloro-4-[(4-oxo-2-thioxo-1,3-thiazolidin-5-ylidene)methyl]phenoxy}benzyl)-3-ethyl-1,3-oxazolidin-2-one
45) 4-(4-{3-chloro-4-[(4-oxo-2-thioxo-1,3-thiazolidin-5-ylidene)methyl]phenoxy}benzyl)-3 -ethyl-1,3-oxazolidin-2-one;
46) 3-ethyl-4-(4-(3-fluoro-4-[(4-oxo-2-thioxo-1,3-thiazolidin-5-ylidene)methyl]phenoxy}benzyl)-1,3-oxazolidin-2-one;
47) 4-(4-{2-methoxy-4-[(3-aceticacid-4-oxo-2-thioxo-1,3-thiazolidin-5-yl)ethyl]phenoxy}-benzyl)-3-methyl-1,3-oxazolidin-2-one;
48) 4-(4-{3-chloro-4-[(3-aceticacid-4-oxo-2-thioxo-1,3-thiazolidin-5-yl)ethyl]phenoxy}-benzyl)-3-methyl-1,3-oxazolidin-2-one;
49) 4-(4-{2-chloro-4-[(3-aceticacid-4-oxo-2-thioxo-1,3-thiazolidin-5-yl)ethyl]phenoxy}-benzyl)-3-ethyl-1,3-oxazolidin-2-one;
50) 4-(4-{2-methoxy-4-[(3-aceticacid-4-oxo-2-thioxo-1,3-thiazolidin-5-yl)ethyl]phenoxy}-benzyl)-3-ethyl-1,3-oxazolidin-2-one;
51) 4-(4-{2-triflouromethyl-51) 4-[(3-acetic acid-4-oxo-2-thioxo-1,3-thiazolidin-5-yl)ethyl]phenoxy)-benzyl)-3-ethyl-1,3-oxazolidin-2-one;
52) 5-(3-fluoro-4-{4-[(3-ethyl-2-oxo-1,3-oxazolidin-4-yl)methyl]phenoxy}benzylidene)-4-oxo-2-thioxo-1,3-thiazolidin-3-yl]acetic acid;
53) [4-oxo-5-(2-chloro-4-{4-[(3-ethyl-2-oxo-1,3-oxazolidin-4-yl)methyl]phenoxy}benzylidene)-2-thioxo-1,3-thiazolidin-3-yl]acetic acid;
54) [(4-oxo-5-(2-fluoro-4-{4-[(3-ethyl-2-oxo-1,3-oxazolidin-4-yl)methyl]phenoxy}benzylidene)-2-thioxo-1,3-thiazolidin-3-yl]acetic acid;
55) 4-(4-{2-triflouromethyl-4-[(4-oxo-2-thioxo-1,3-thiazolidin-5-yl)methyl]phenoxy}benzylidene)-3-methyl-1,3-oxazolidin-2-one;
56) [4-oxo-5-(3-trifluoromethyl-4-{4-[(3-methyl-2-oxo-1,3-oxazolidin-4-yl)methyl]phenoxy}benzylidene)-2-thioxo-1,3-thiazolidin-3-yl]acetic acid;
57) [(4-oxo-5-(2-fluoro-4-{4-[(3-methyl-2-oxo-1,3-oxazolidin-4-yl)methyl]phenoxy}benzylidene)-2-thioxo-1,3-thiazolidin-3-yl]acetic acid;
58) 4-(4-{3-chloro-4-[(4-oxo-2-thioxo-1,3-thiazolidin-5-ylidene)methyl]phenoxy}benzyl)-3-methyl-1,3-oxazolidin-2-one;
59) 4-(4-{2-triflouromethyl-4-[(4-oxo-2-thioxo-1,3-thiazolidin-5-yl)methyl]phenoxy}benzylidene)-3-ethyl-1,3-oxazolidin-2-one;
60) 4-(4-{2-chloro-4-[(4-oxo-2-thioxo-1,3-thiazolidin-5-ylidene)methyl]phenoxy}benzyl)-1,3-oxazolidin-2-one;
61) 5-(4-{4-[(3-methyl-2-oxo-1,3-oxazolidin-4-yl)methyl]phenoxy}benzyl)-1,3-thiazolidine-2,4-dione;
62) 5-(4-{4-[(3-ethyl-2-oxo-1,3-oxazolidin-4-yl)methyl]phenoxy}benzyl)-1,3-thiazolidine-2,4-dione;
63) 5-(3-fluoro-4-{4-[(3-methyl-2-oxo-1,3-oxazolidin-4-yl)methyl]phenoxy}benzyl)-1,3-thiazolidine-2,4-dione;
64) 5-(4-{4-[(3-methyl-2-oxo-1,3-oxazolidin-4-yl)methyl]phenoxy}benzyl)-1,3-dihydro-2H-indol-2-one;
65) 5(3-trifluoromethyl-(4-{4-[(3-ethyl-2-oxo-1,3-oxazolidin-4-yl)methyl]phenoxy}benzyl)-1,3-dihydro-2H-indol-2-one;
66) 5(3-fluoro-(4-{4-[(3-ethyl-2-oxo-1,3-oxazolidin-4-yl)methyl]phenoxy}benzyl)-1,3-dihydro-2H-indol-2-one;
67) 5(3-methoxy-(4-{4-[(3-methyl-2-oxo-1,3-oxazolidin-4-yl)methyl]phenoxy}benzyl)-1,3-dihydro-2H-indol-2-one
68) 5-(4-{4-[(3-ethyl-2-oxo-1,3-oxazolidin-4-yl)methyl]phenoxy}benzyl)-1,3-dihydro-2H-indol-2-one;
69) 5(3-methoxy-(4-{4-[(3-ethyl-2-oxo-1,3-oxazolidin-4-yl)methyl]phenoxy}benzyl)-1,3-dihydro-2H-indol-2-one;
70) 5-(2-chloro-4-{4-[(3-methyl-2-oxo-1,3-oxazolidin-4-yl)methyl]phenoxy}benzyl)-1,3-thiazolidine-2,4-dione;
71) 4-{4-[(3-methyl-2-oxo-1,3-oxazolidin-4-yl)methyl]phenoxy)-3-(trifluoromethyl benzyl]-1,3-thiazolidine-2,4-dione;
72) 3-chloro-4-{4-[(3-methyl-2-oxo-1,3-oxazolidin-4-yl)methyl]phenoxy}benzyl)-1,3-thiazolidine-2,4-dione;
73) 5-(3-chloro-4-{4-[(3-ethyl-2-oxo-1,3-oxazolidin-4-yl)methyl]phenoxy}benzyl)-1,3-thiazolidine-2,4-dione;
74) 5-(3-methoxy-4-{4-[(3-methyl-2-oxo-1,3-oxazolidin-4-yl)methyl]phenoxy}benzyl)-1,3-thiazolidine-2,4-dione;
75) 5-(3-trifluoromethyl-(4-{4-[(3-methyl-2-oxo-1,3-oxazolidin-4-yl)methyl]phenoxy}benzyl)-1,3-dihydro-2H-indol-2-one;
76) 5-(4-{4-[(3-methyl-2-oxo-1,3-oxazolidin-4-yl)methyl]phenoxy}benzyl)-2-thioxo-1,3-thiazolidin-3-yl]acetic acid;
77) 3-methyl-4-(4-{4-[(4-oxo-2-thioxo-1,3-thiazolidin-5-yl)methyl]phenoxy}benzyl)-1,3-oxazolidin-2-one;
78) 3-ethyl-4-(4-{4-[(4-oxo-2 -thioxo-1,3-thiazolidin-5-yl)methyl]phenoxy}benzyl)-1,3-oxazolidin-2-one;
79) 5-(4-{4-[(3-ethyl-2-oxo-1,3-oxazolidin-4-yl)methyl]phenoxy}benzyl)-2-thioxo-1,3-thiazolidin-3-yl]acetic acid;
80) 4-(4-{2-fluoro-4-[(4-oxo-2-thioxo-1,3-thiazolidin-5-yl)methyl]phenoxy}benzyl)-3-methyl-1,3-oxazolidin-2-one;
81) 5-(3-fluoro-4-{4-[(3-methyl-2-oxo-1,3-oxazolidin-4-yl)methyl]phenoxy}benzyl)-4-oxo-2-thioxo-1,3-thiazolidin-3-yl]acetic acid;
82) 4-(4-{2-chloro-4-[(4-oxo-2-thioxo-1,3-thiazolidin-5-yl)methyl]phenoxy}benzyl)-3-methyl-1,3-oxazolidin-2-one;
83) 4-(4-{2-chloro-4-[(4-oxo-2-thioxo-1,3-thiazolidin-5-yl)methyl]phenoxy}benzyl)-3-ethyl-1,3-oxazolidin-2-one;
84) 4-(4-{2-fluoro-4-[(4-oxo-2-thioxo-1,3-thiazolidin-5-yl)methyl]phenoxy}benzyl)-N-ethyl-1,3-oxazolidin-2-one;
85) 4-{4-[4-[(4-oxo-2-thioxo-1,3-thiazolidin-5-yl)methyl]-2-(trifluoromethyl)phenoxy]benzyl}-N-ethyl-1,3-oxazolidin-2-one;
86) 5-(3-trifluoromethyl-4-(4-[(3-methyl-2-oxo-1,3-oxazolidin-4-yl)methyl]phenoxy}benzyl)-4-oxo-2-thioxo-1,3-thiazolidin-3-yl]acetic acid;
87) 5-(3-trifluoromethyl-4-(4-[(3-ethyl-2-oxo-1,3-oxazolidin-4-yl)methyl]phenoxy}benzyl)-4-oxo-2-thioxo-1,3-thiazolidin-3-yl]acetic acid;
88) 5-(2-chloro-4-{4-[(3-ethyl-2-oxo-1,3-oxazolidin-4-yl)methyl]phenoxy}benzyl)-4-oxo-2-thioxo-1,3-thiazolidin-3-yl]acetic acid;
89) 4-{4-[4-[(4-oxo-2-thioxo-1,3-thiazolidin-5-yl)methyl]-2-(trifluoromethyl)phenoxy]benzyl}-N-methyl-1,3-oxazolidin-2-one
90) 5-(3-chloro-4-{4-[(3-methyl-2-oxo-1,3-oxazolidin-4-yl)methyl]phenoxy}benzyl)-4-oxo-2-thioxo-1,3-thiazolidin-3-yl]acetic acid
91) 4-(4-{3-fluoro-4-[(4-oxo-2 -thioxo-1,3-thiazolidin-5-yl)methyl]phenoxy}benzyl)-3-methyl-1,3-oxazolidin-2-one and
92) 4-(4-{3-chloro-4-[(4-oxo-2-thioxo-1,3-thiazolidin-5-yl)methyl]phenoxy}benzyl)-3-methyl-1,3-oxazolidin-2-one.

Preferred salts for the list of compounds above are hydrochloride, hydrobromide, sodium, potassium or magnesium.

According to another feature of the present invention, there is provided a process for the preparation of the compounds of Formula (1) wherein [symbol] represents a bond and all others are as defined earlier as shown in scheme-I

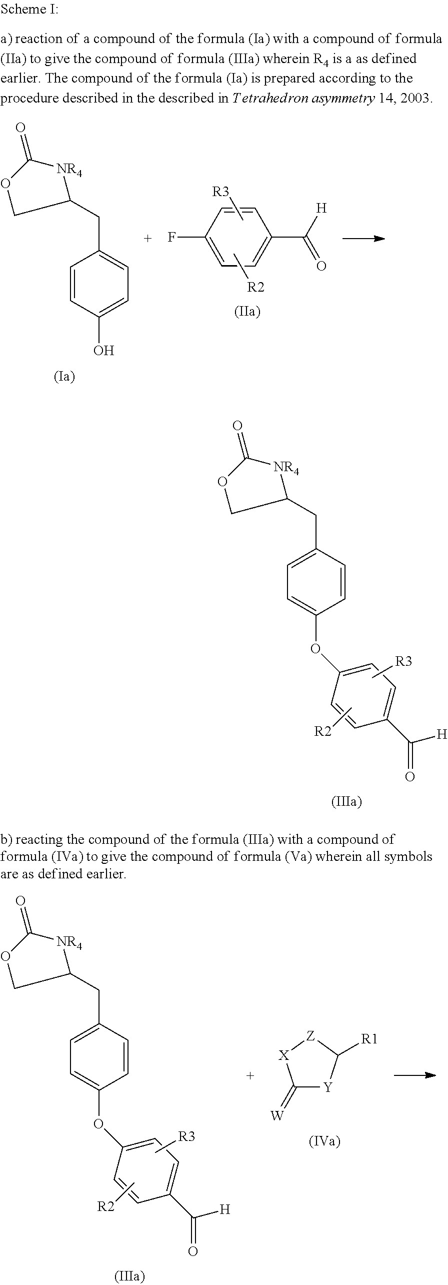

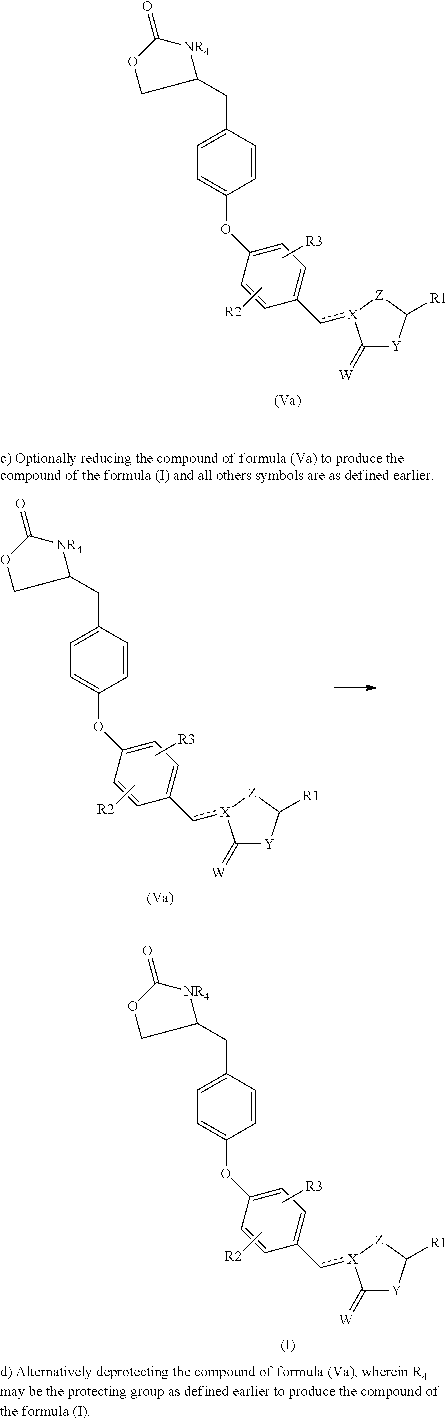

The reactions described in the processes outlined above is performed by using the methods described herein:

### Scheme-I

The reaction of compound of formula (Ia) with the compound of formula (IIa) produce a compound of formula (IIIa) in the presence of solvents such as tetrahydrofuran, dimethylformamide, dimethyl sulfoxide, and the like or mixtures of solvents may be used. The reaction may be carried out in an inert atmosphere. The reaction may be effected in the presence of a base such as K₂CO₃, Na₂CO₃, NaH or mixtures thereof. The reaction temperature may range from 60° C. to 150° C., preferably at a temperature in the range of 80° C. to 100° C. The duration of the reaction may range from 1 to 24 hrs, preferably from 2 to 6 hrs. The reaction of the compound of the formula (IIIa) with a compound of formula (IVa) is carried out in the presence of base and in the presence of a solvent such as toluene, methoxyethanol or mixtures thereof to yield a compound of formula (Va). The reaction temperature may range from 60° C. to 180° C. Suitable catalyst such as piperidinium acetate or benzoate, sodium acetate or mixtures of catalysts may also be employed. The water produced in the reaction may be removed by using Dean Stark water separator or by using water-absorbing agents like molecular sieves.

The deprotection of formula (Va) to yield compound of formula (I) may be carried out using acids such as HCl, sulfuric acid, acetic acid in the presence of solvents such as dichloromethane, ethyl acetate, water and the like or mixture thereof at a temperature in the range of −10° C. to 50° C.

In another embodiment of the present invention, there is provided a process for the preparation of compounds of formula (I), by reducing the penultimate step of formula (I) wherein [symbol] represents bond The reduction step is not required when represent and all other symbols are as defined earlier. The reduction may be carried out in the presence of gaseous hydrogen and a catalyst such as Pd/C, Rh/C, Pt/C, Raney Nickel, and the like. Mixtures of catalysts may be used. The reaction may be conducted in the presence of solvents such as methanol, dichloromethane, dioxane, acetic acid, ethyl acetate and the like. Mixtures of solvents may be used. A pressure between atmospheric pressure to 100 psi may be employed. The catalyst may be 5-10% Pd/C and the amount of catalyst used may range from 50-300% w/w.

The order of doing deprotection and reduction can be changed or reversed.

The invention is explained in detail in the examples given below which are provided by way of illustration only and therefore should not be construed to limit the scope of the invention.

### Example 1

### Preparation of (5E)-5-(4-{4-[(3-methyl-2-oxo-1,3-oxazolidin-5-l)methyl]phenoxy}benzylidene)-1,3-thiazolidine-2,4-dione

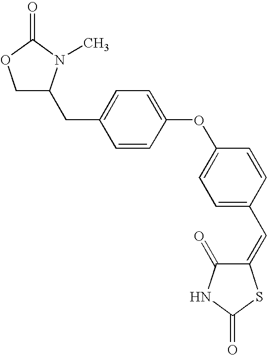

### Step I

### Synthesis of 4-{4-[(3-methyl-2-oxo-1,3-oxazolidin-4-yl)methyl]phenoxy}benzaldehyde

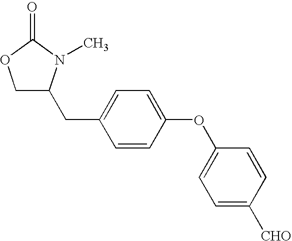

To the suspension of potassium carbonate (13.33 g, 96.6 mmol) in dry dimethylformamide (20 ml), charged 4-(4-hydroxybenzyl)-3-methyl-1,3-oxazolidin-2-one (2.0 g, 9.66 mmol), (Tetrahedron asymmetry 14, 2003, 2619-2623) at 30° C. Reaction mixture was stirred for 15 min and charged p-fluorobenzaldehyde (2.39 g, 19.32 mmol). The reaction mixture was warmed to 80° C. and stirred for 24 hr. The reaction mixture was quenched by water and extracted with ethyl acetate. Combined organic layer was dried over sodium sulfate and concentrated, purified to yield 4-{4-[(3-methyl-2-oxo-1,3-oxazolidin-4-yl)methyl]phenoxy}benzaldehyde. Yield: 2.9 g (97.6%).

### Step II

### Synthesis of (5E)-5-(4-{4-[(3-methyl-2-oxo-1,3-oxazolidin-5-l)methyl]phenoxy}benzylidene)-1,3-thiazolidine-2,4-dione

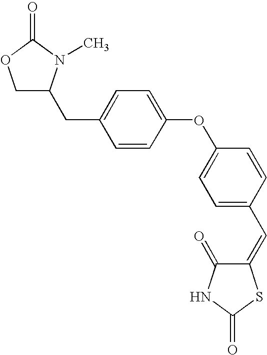

To the suspension of 4-{4-[(3-methyl-2-oxo-1,3-oxazolidin-4-yl)methyl]phenoxy}benzaldehyde (1.5 g, 4.82 mmol) and toluene (50 ml) charged 2,4-thiazolidinonedione (0.62 g, 5.30 mmol), benzoic acid (0.088 g, 0.72 mmol) and piperidine (0.053 g, 0.62 mmol). The reaction mixture was refluxed at 145°-155° C. with continuous removal of water using dean stark apparatus for 3 hr. The solvent was removed by distillation. The crude product thus obtained was purified to Yield: 1.9 g (96.4%, ¹H NMR DMSO-d₆ 400 MHz): δ 2.7 (m, 1H), 2.79 (s, 3H), 3.08 (m, 1H), 3.9 (m, 2H), 4.2 (m, 1H), 7.0 (m, 4H), 7.3 (d, 2H), 7.6 (d, 2H), 7.7 (s, 1H); m/z^{M+1}: 410.9.

### The Following Compound were Prepared According to the Procedure Give in Example 1

| Example | Structure | Analytical Data |
|---|---|---|
| 2 | 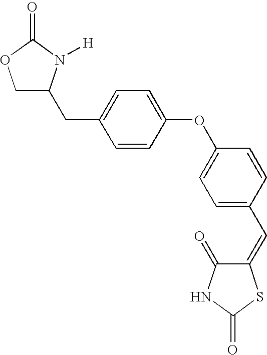 | Yieid: 0.155 g, (74.8%, ¹HNMR CDCl₃, 400 MHz): δ 2.8 (d. 2H), 4.0 (m, 2H), 4.3 (t, 1H), 7.0 (m, 4H), 7.3 (d, 2H), 7.6 (d, 2H), 7.7 (s, 1H) , 7.79 (s, 1H) , 12.58 (s, 1H). m/z^{M+l}: 397.1 |
| 3 | 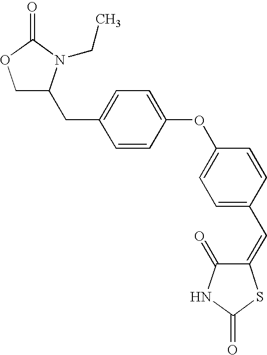 | Yield: 1.25 g (96.15%, ¹HNMK (CDCl₃ 400 MHz): δ 1.2 (t, 3H), 2.7 (m, 1H), 3.1 (m, 2H), 3.6 (m, 1H), 4.0 (q, 2H), 4.2 (m, 1H), 7.0 (t, 4H), 7.2 (d, 2H), 7.4 (d, 2H), 7.8 (s, 1H), 8.61 (bs, 1H); m/z^{M+l}: 425.1 |
| 4 | 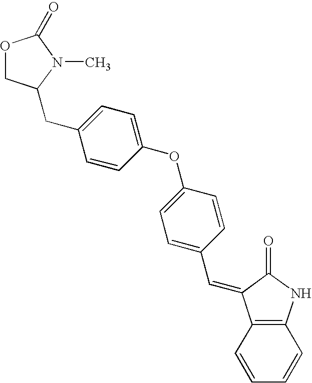 | Yield: 0.15 g (56.19%, ¹HNMR CDCl₃ 400 MHz): δ 2.7 (m, 1H), 2.9 (s, 3H), 3.1 (dd, 1H), 3.9 (m, 1H), 4.0 (m, 1H), 4.2 (t, 1H), 6.8 (d, 1H), 7.0 (m, 5H), 7.1 (m, 3H), 7.5 (m, 3H), 8.3 (d, 2H); m/z^{M+1}: 427.1. |
| 5 | 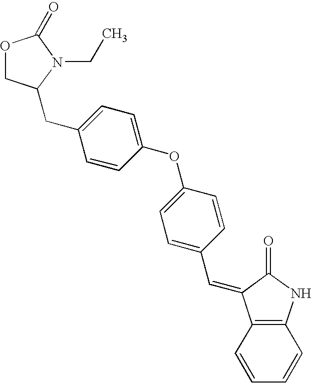 | Yield: 0.12 g (65.95%, ¹HNMR CDCl₃ 400 MHz): δ 1.2 (t, 3H), 2.6 (m, 1H), 3.1 (m, 2H), 3.6 (m, 1H), 4.0 (q, 2H), 4.2 (m, 1H), 6.8 (m, 2H), 7.0 (m, 4H), 7.1 (m, 3H), 7.6 (m, 3H), 7.7 (s, 1H), 7.9 (s, 1H); m/z^{M+l}: 441.3. |
| 6 | 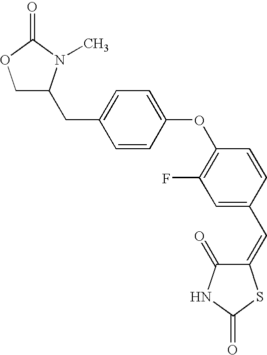 | Yieid: 0.605 g (46.15%, ¹HNMR CDCl₃ CDCl3, 400 MHz): δ 2.7 (m, 1H), 2.9 (s, 3H), 3.1 (dd, 1H), 3.9 (m, 2H), 4.2 (t, 1H), 7.0 (t, 3H), 7.1 (d, 2H), 7.2 (dd, 1H), 7.3 (s, 1H), 7.7 (s, 1H). m/z^{M+1}: 429.2. |
| 7 | 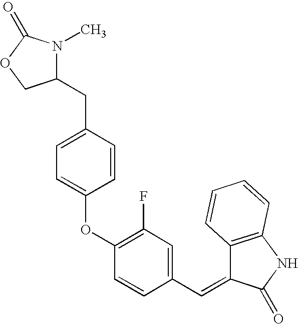 | Yield: 0.62 g (57.40%, ¹HNMR CDCl₃- 400 MHz): δ 2.7 (m, 1H), 2.9 (s, 3H), 3.1 (dd, 1H), 3.9 (m, 1H), 4.0 (t, 1H), 4.2 (t, 1H), 6.8 (m, 1H), 7.0 (m, 3H), 7.1 (m, 2H), 7.2 (m, 1H), 7.4 (s, 1H), 7.5 (d, 1H), 7.6 (m, 2H), 7.9 (d, 1H). m/z^{M+1}: 445.1 |
| 8 | 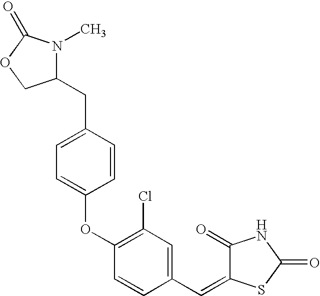 | Yield: 0.l g (78.90%, 1HNMR CDCl₃-400 MHz): δ 2.72 (m; 1H), 2.92 (s, 3H), 3.13 (dd, 1H), 3.9 (m, 1H), 3.97 (t, 1H), 4.24 (t, 1H), 7.0 (m, 3H), 7.2 (m, 2H), 7.32 (dd, 1H), 7.8 (s, 1H), 7.72 (s, 1H), 8.0 (s, 1H), m/zM+1: 445.0 |
| 9 | 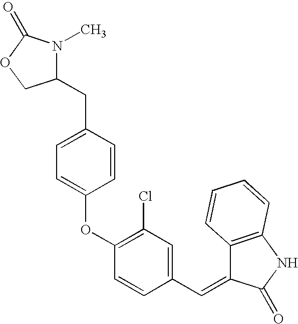 | Yield: 0.82 g (61.65% , ¹HNMR CDCl₃ 400 MHz): δ 2.7 (m, 1H), 2.9 (s, 3H), 3.1 (dd, 1H), 3.9 (m, 1H), 4.0 (t, 1H), 4.2 (t, 1H), 6.99 (m, 5H), 7.18 (m, 3H), 7.4 (s, 1H), 7.5 (m, 2H), 7.69 (s, 1H), 8.46 (s, 1H); m/z^{M+l}: 461.2. |
| 10 | 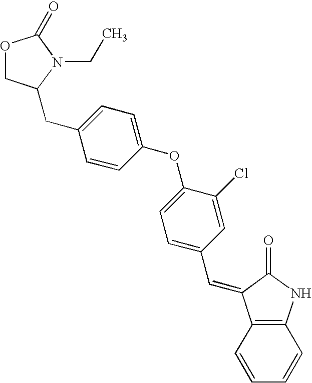 | Yield: 0.85 g (64.8% , ¹HNMR CDCl₃ 400 MHz): δ 1.20 (t, 3H), 3.16 (m, 2H), 3.62 (m, 2H), 4.02 (q, 2H), 4.2 (m, 1H), 6.85 (d, 1H), 6.93 (d, 1H), 7.05 (m, 3H), 7.16 (d, 2H), 7.24 (m, 1H), 7.42 (s, 1H), 7.50 (d, 2H), 8.3 (dd, 1H), 8.4 (d, 1H); m/z^{M+1}: 475, 476.7 |
| 11 | 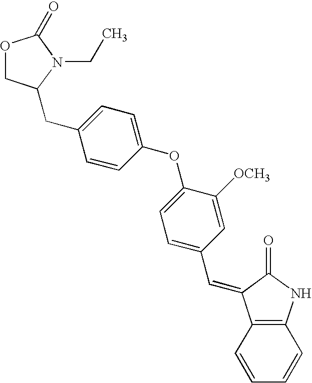 | Yield: 0.85 g (63.9% , ¹HNMR CDCl₃ 400 MHz): δ 2.73 (m, 1H), 2.89 (s, 3H), 3.10 (dd, 1H), 3.87 (s, 3H), 4.01 (m, 1H), 4.21 (t, 1H), 6.90 (m, 2H), 7.01 (m, 3H), 7.13 (m, 2H), 7.21 (m, 1H), 7.31 (s, 2H), 7.50 (m, 2H), 7.77 (d, 1H); m/z^{M+l}: 457.2 |
| 12 | 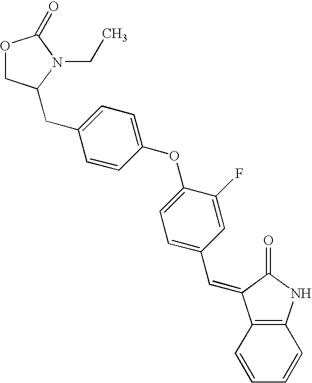 | Yield: 0.33 g (30.89% , ¹HNMR CDCl₃ 400 MHz): δ 1.20 (t. 3H), 2.7 (m, 1H), 3.15 (m, 2H), 3.62 (m, 1H), 4.02 (q, 2H), 4.2 (m, 1H), 6.85 (d, 1H), 7.02 (m, 4H), 7.16 (m, 2H), 7.25 (s, 1H), 7.44 (s, 1H), 7.52 (d, 1H), 7.60 (s, 1H), 7.80 (dd, 1H), 8.51 (dd, 1H); m/z^{M+1}: 459.2 |
| 13 | 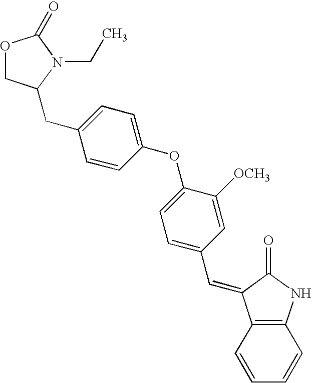 | Yield: 0.59 g (49.57% , ¹HNMR CDCl₃ 400 MHz): δ 1.20 (t, 3H), 2.7 (m, 1H), 3.15 (m, 2H), 3.60 (m, 1H), 3.88 (s,,3H), 4.02 (q, 2H), 4.2 (m, 1H), 6.90 (m, 2H), 7.02 (m, 3H), 7.14 (m, 2H), 7.27 (m, 1H), 7.31 (s, 1H), 7.76 (m, 1H), 7.78 (m, 2H); m/z^{M+1}: 471.2 |
| 14 | 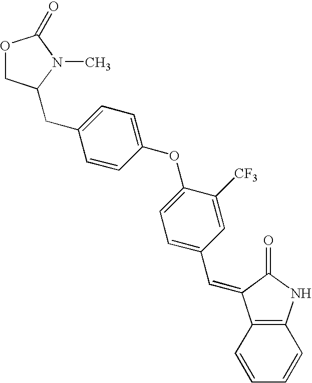 | Yield: 0.23 g (49.5% , ¹HNMR DMSO-d₆ 400 MHz): δ 2.77 (m, 1H), 2.80 (s, 3H), 3.06 (dd, 1H), 3.98 (m, 2H), 4.20 (m, 1H), 6.84 (d, 1H), 7.01 (m, 2H), 7.11 (d, 2H), 7.23 (m, 1H), 7.37 (d, 2H), 7.70 (d, 1H), 7.88 (s, 1H), 8.51 (d, 1H), 9.1 (s, 1H), 10.6 (s, 1H); m/z^{M+1}: 495.2 |
| 15 | 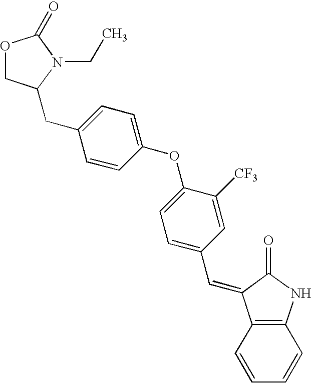 | Yield: 1.0 g (83.3% , ¹HNMR, DMSO-d₆ 400 MHz): δ 1.09 (t, 3H), 2.77 (m, 1H), 3.12 (m, 2H), 3.38 (m, 1H), 3.98 (m, 1H), 4.2 (q, 2H), 6.84 (d, 1H), 6.99 (d, 2H), 7.11 (d, 2H), 7.21 (d, 1H), 7.39 (d, 2H), 7.70 (d, 1H), 7.88 (s, 1H), 8.51 (d, 1H), 9.1 (s, 1H);m/z^{M+1}: 509.2 |
| 16 | 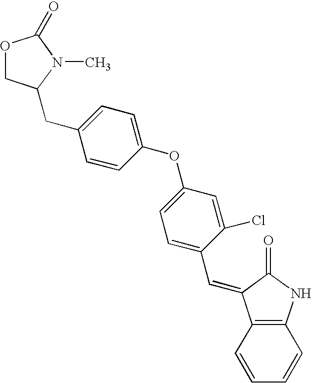 | Yield: 0.45 g (33.8% , ¹HNMR, CDCl₃ 400 MHz): δ 2.73 (m, 1H), 2.93 (s, 3H), 3.12 (dd, 1H), 3.94 (m, 1H), 4.03 (q, 1H), 4.25 (m, 1H), 6.86 (m, 3H), 7.08 (m, 4H), 7.18 (m, 3H), 7.77 (d, 1H), 7.83 (d, 1H); m/z^{M+1}: 461.1 |
| 17 | 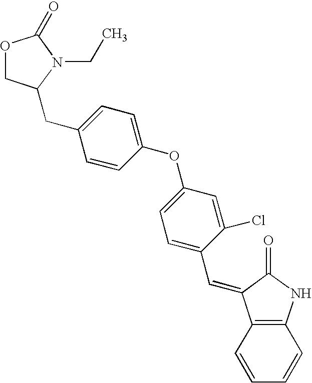 | Yield: 0.89 g (67.4% , ¹HNMR, CDCl₃, 400 MHz): δ 1.08 (t, 3H), 2.9 (m, 1H), 3.12 (m, 2H), 3.36 (m, 2H), 3.98 (t, 1H), 4.17 (m, 2H), 6.88 (m, 2H), 7.05 (d, 1H), 7.20 (d, 2H), 7.29 (m, 1H), 7.31 (d, 1H), 7.53 (s, 1H), 7.82 (d, 1H), 10.67 (s, 1H); m/z^{M+1}: 475.1 |
| 18 | 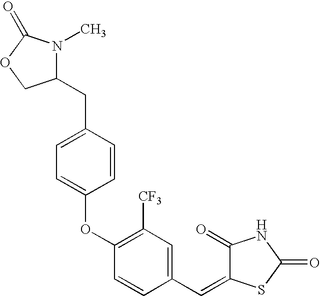 | Yield: 0.97 g (74.4% , ¹HNMR DMSO d₆-400 MHz): δ 2.76 (m, 1H), 2.79 (s, 3H), 3.07 (dd, 1H), 3.96 (m, 2H), 4.20 (t, 1H), 7.07 (d, 3H), 7.12 (d, 2H), 7.36 (d, 2H), 7.79 (t, 1H), 8.05 (s, 1H); m/z^{M+l}: 479 |
| 19 | 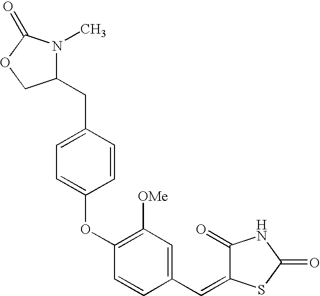 | Yield: 0.39 g (30.23% , ¹HNMR CDCl₃-400 MHz): δ 2.73 (m, 1H), 2.79 (s, 3H), 3.07 (dd, 1H), 3.92 (s. 3H), 3.95 (m, 1H), 4.00 (t, 1H), 4.23 (t, 1H), 6.96 (m, 3H), 7.12 (m, 4H), 7.81 (s, 1H); m/z^{M+1}: 441.1 |
| 20 | 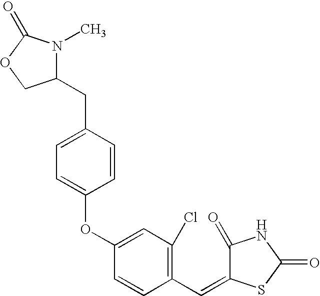 | Yield: 0.4 g (62.17 % , ¹HNMR CDCl₃-400 MHz): δ 2.76 (m, 1H), 2.99 (s, 3H), 3.14 (dd, 1H), 3.95 (m, 1H), 4.02 (t, 1H), 4.26 (t, 1H), 6.94 (dd, 1H), 7.06 (m, 3H), 7.20 (m, 2H), 7.48 (d, 1H), 8.15 (s, 1H); m/z^{M+1}: 445.1 |
| 21 | 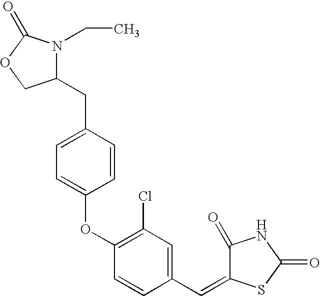 | Yield: 1.17 g (87.4% , ¹HNMR CDCl₃-400 MHz): δ 1.20 (t, 3H), 2.72 (m, 1H), 3.16 (m, 2H), 3.61 (m, 1H), 4.03 (q, 2H), 4.20 (m, 1H), 6.94 (d, 1H), 7.02 (d, 2H), 7.20 (m, 3H), 7.60 (s, 1H), 7.74 (s, 1H),; m/z^{M+1}: 459.2 |
| 22 | 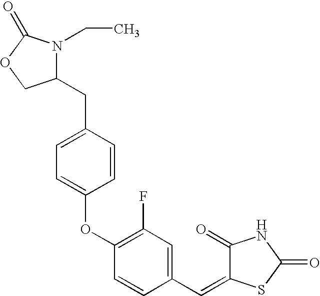 | Yield: 0.60 g (46.87%, ¹HNMR CDCl₃-400 MHz): δ 1.20 (t, 3H), 2.73 (m, 1H), 3.12 (dd, 1H), 3.17 (m, 1H), 3.61 (m, 1H), 4.03 (q, 2H), 4.18 (t, 1H), 7.02 (m, 3H), 7.18 (m, 3H), 7.34 (dd, 1H), 7.74 (s, 1H),; m/z^{M+1}: 443.1 |
| 23 | 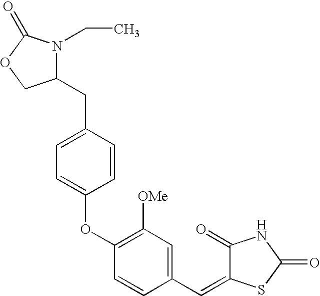 | Yield: 0.74 g (58.25% , ¹HNMR CDCl₃-400 MHz): δ 1.2 (t, 3H), 2.68 (m, 1H), 3.08 (dd, 1H), 3.15 (m, 1H), 3.61 (m, 1H), 3.92 (s, 3H), 4.03 (q, 2H), 4.20 (m, 1H), 6.94 (m, 3H), 7.08 (m, 4H), 7.81 (s, 1H),; m/z^{M+1}: 455.2 |
| 24 | 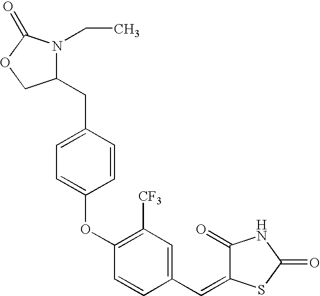 | Yield: 0.800 g (72.7%, ¹HNMR DMSO d₆-400 MHz): δ 1.09 (t, 3H), 2.77 (m, 1H), 3.05 (dd, 1H), 3.15 (m, 1H), 3.38 (m, 1H), 3.98 (dd, 3H), 4.18 (m, 3H), 7.04 (d, 1H), 7.08 (d, 2H), 7.37 (d, 2H), 7.79 (dd, 1H), 7.85 (s, 1H), 8.03 (d, 1H); m/z^{M+l}: 493.3 |
| 25 | 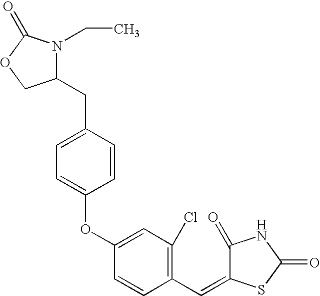 | Yield: l.00 g (78.7% , ¹HNMR CDCl₃-400 MHz): δ 1.21 (t, 3H), 2.75 (m, 1H), 3.16 (m, 2H), 3.62 (m, 1H), 4.01 (m, 2H), 4.20 (t, 3H), 6.94 (dd, 1H), 7.06 (m, 2H), 7.22 (m, 3H), 7.46 (d, 1H), 8.16 (s, 1H); m/z^{M+l}: 459.2 |
| 26 | 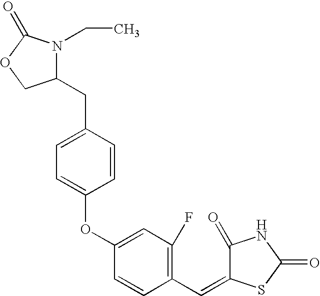 | Yield: 0.250 g (19.53%, ¹HNMR CDCl₃-400 MHz): δ 1.21 (t, 3H), 2.72 (m, 1H), 3.16 (m, 2H), 3.62 (m, 1H), 4.01 (m, 2H), 4.22 (t, 3H), 6.71-8.19 (m, 8H), 8.68 (bs, 1H) ; m/z^{M+1}: 459.2 |
| 27 | 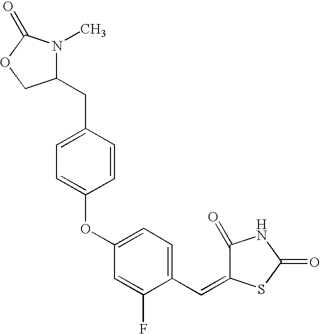 | Yield: 0.52 g (40.0%, ¹HNMR CDCl₃-400 MHz): δ 2.76 (m, 1H), 2.93 (s, 3H), 3.14 (dd, 1H), 3.96 (m, 2H), 4.02 (t, 1H), 4.28 (t, 1H), 6.74 (d, 1H), 6.86 (d, 1H), 7.00 (m, 2H), 7.23 (m, 2H), 7.4 (t, 1H), 8.0 (s, 1H); m/z^{M+l}: 429.2 |
| 28 | 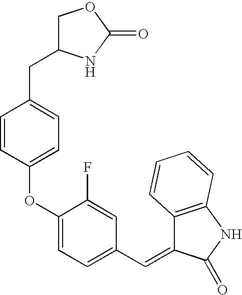 | Yield: 0.080 g (12.32%, ¹HNMR CDCl₃-400 MHz): 2.78 (d, 2H), 4.06 (m, 2H), 4.29 (m, 1H), 6.89 (d, 2H), 7.06 (d, 2H), 7.17 (m, 1H), 7.24 (m, 1H), 7.31 (m, 2H), 7.57 (s, 1H), 7.59 (s, 1H), 7.76 (m, 2H), 10.6 (s, 1H); m/z^{M+l}: 431.1 |
| 29 | 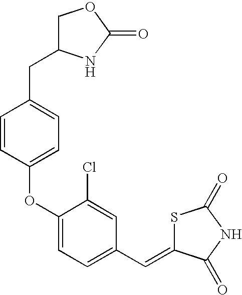 | Yield: 0.300 g (61.7%, ¹HNMR CDCl₃-400 MHz): 2.80 (d, 2H), 4.04 (m, 2H), 4.33 (t, 1H), 7.06 (m, 3H), 7.32 (d, 2H), 7.52 (m, 1H), 7.76 (s, 1H), 7.83 (s, 1H); m/z^{M+1}: 431 |
| 30 | 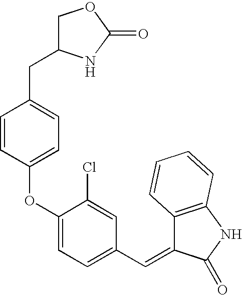 | Yield: 1.0 g (66.6%, ¹HNMR CDCl₃-400 MHz): 2.78 (m, 2H), 4.04 (m, 2H), 4.30 (m, 1H), 6.89 (d, 2H), 7.06 (d, 3H), 7.23 (m, 3H), 7.54 (m, 1H), 7.69 (d, 1H), 7.93 (s, 1H), 10.65 (s, 1H); m/z^{M+1}: 447 |
| 31 | 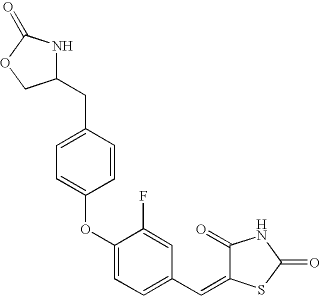 | Yield: 0.400 g (74.07%, ¹HNMR CDCl₃-400 MHz): 2.87 (m, 2H), 4.14 (m, 1H), 4.16 (t, 1H), 4.48 (t, 1H), 7.03 (m, 3H), 7.20 (m, 3H), 7.31 (m, 1H), 7.74 (s. 1H); m/z^{M+l}: 415 |

### Example 32

### 3-methyl-5-(4-{4-[(E)-(3-aceticacid-4-oxo-2-thioxo-1,3-thiazolidin-5-ylidene)methyl]phenoxy}benzyl)-1,3-oxazolidin-2-one

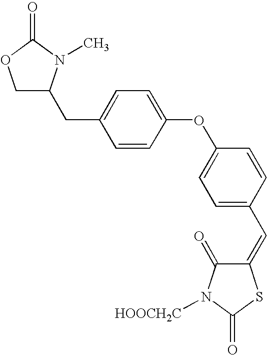

A solution of 4-{4-[(3-methyl-2-oxo-1,3-oxazolidin-4-yl)methyl]phenoxy}benzaldehyde (Example 1, Step I) (1.0 g, 3.21 mmol), rhodanine-3-acetic acid (0.674 g, 3.53 mmol), benzoic acid (0.058 g, 0.47 mmol) and piperidine (0.035 g, 0.41 mmol) in toluene (40 ml) was refluxed with stirring at 145°-155° C. with continuous removal of water using dean stark apparatus for 3 hr. The reaction mixture was allowed to attain 30° C. and concentrated. The crude product thus obtained was purified to desire product Yield: 1.42 g (91.6%, ¹HNMR DMSO-d₆ 400 MHz): δ 2.6 (m, 1H), 2.7 (s, 3H), 3.0 (dd, 1H), 3.9 (m, 2H), 4.1 (m, 1H), 4.7 (s, 2H), 7.1 (dd, 4H), 7.3 (d, 2H), 7.6(d, 2H), 7.8(s, 1H), 12.7 (bs, 1H); m/z^{M+1}: 484.9.

### The Following Compound were Prepared According to the Procedure Give in Example 32

| Example | Structure | Analytical data |
|---|---|---|
| 33 | 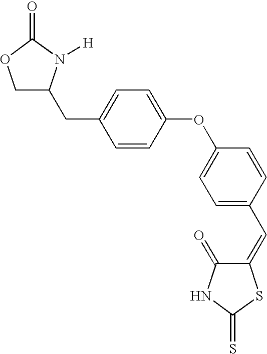 | Yield: 0.097 g (75%, ¹HNMR CDCl₃ 400 MHz): δ 2.8 (m, 1H), 3.4 (m, 2H), 4.1 (m, 1H), 4.5 (m, 1H), 7.0 (m, 4H), 7.2 (d, 2H), 7.4 (d, 2H), 7.6 (s, 1H); m/z^{M+1}: 413. |
| 34 | 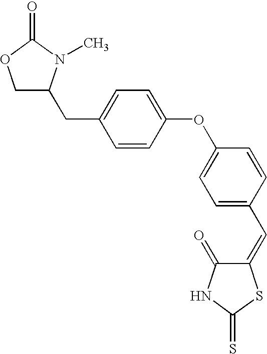 | Yield: 1.25 g (76.2%, ¹HNMR CDCl₃ 400 MHz): δ 2.7 (m, 1H), 2.93 (s, 3H), 3.1 (dd, 1H), 3.9 (m, 1H), 4.0 (q, 1H), 4.2 (t, 1H), 7.0 (t, 4H), 7.1 (d, 2H), 7.4 (d, 2H), 7.6 (s, 1H). m/z^{M+1}: 427. |
| 35 | 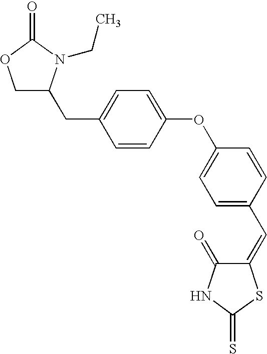 | Yield: 0.940 g (69.29%, ¹HNMR CDCl₃ 400 MHz): δ 1.2 (t, 3H), 2.7 (m, 1H), 3.1 (m, 2H), 3.6 (m, 1H), 4.0 (q, 2H), 4.2 (m, 1H), 7.0 (t, 4H), 7.2 (d, 2H), 7.4 (d, 2H), 7.6 (s, 1H), 9.69 (bs, 1H); m/z^{M+1}: 441.2 |
| 36 | 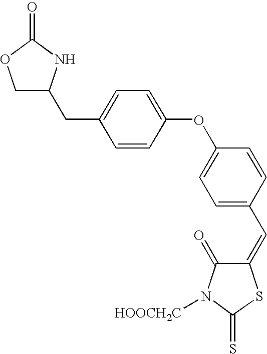 | Yield: 0.155 g (75.24%, ¹HNMR DMSO-d₆ 400 MHz): δ 2.8 (d, 2H), 4.0 (m, 2H), 4.1 (t, 1H), 4.7 (s, 2H), 7.1 (q, 4H), 7.3 (d, 2H), 7.6 (d, 2H), 7.8 (s, 1H), 7.89 (s, 1H); m/z^{M+1}: 471 |
| 37 | 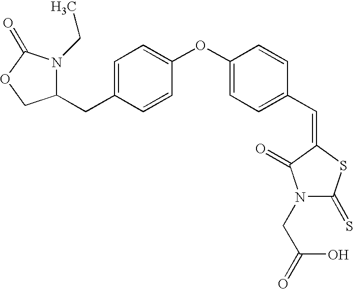 | Yield: 0.92 g (73.77%, ¹HNMR CDCl₃ 400 MHz): δ 1.2 (t, 3H), 2.6 (m, 1H), 3.1 (m, 2H), 3.6 (m, 1H), 4.0 (q, 2H), 4.2 (m, 1H), 4.8 (s, 2H), 7.0 (m, 4H), 7.1 (d, 2H), 7.4 (d, 2H), 7.7 (s, 1H); m/z^{M+1}: 499.5 |
| 38 | 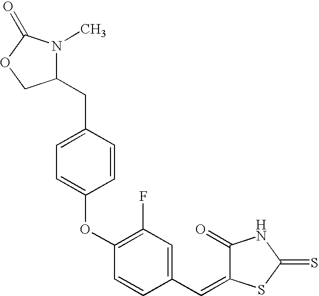 | Yield: 0.68 g (50.74%, ¹HNMR CDCl₃-400 MHz ): δ 2.7 (m, 1H), 2.9 (s, 3H), 3.1 (dd, 1H), 3.9 (m, 1H), 4.0 (m, 1H), 4.2 (t, 1H), 7.0 (m, 3H), 7.1 (m, 3H), 7.3 (m, 1H), 7.5 (s, 1H). m/z^{M+1}: 445.1 |
| 39 | 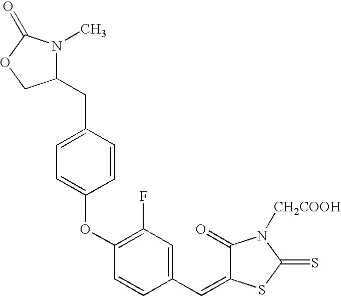 | Yield: 0.35 g (46.05%, ¹HNMR CDCl₃ 400 MHz): δ 2.7 (m, 1H), 2.9 (s, 3H), 3.1 (dd, 1H), 3.9 (m, 1H), 4.0 (t, 1H), 4.2 (t, 1H), 4.9 (s, 2H), 7.0 (m, 3H), 7.1 (m, 3H), 7.3 (m, 1H), 7.6 (s, 1H); m/z^{M+1}: 503.1. |
| 40 | 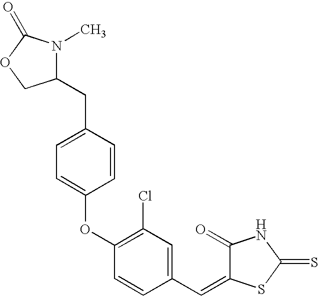 | Yield: 1.07 g (80.45%, ¹HNMR CDCl₃-400 MHz): δ 2.7 (m, 1H), 2.9 (s, 3H), 3.1 (dd, 1H), 3.9 (m, 1H), 3.96 (t, 1H), 4.25 (t, 1H), 6.98 (m, 3H), 7.21 (m, 2H), 7.3 (m, 1H), 7.59 (s, 1H), 7.68 (s, 1H); m/z^{M+1}: 461.1 |
| 41 | 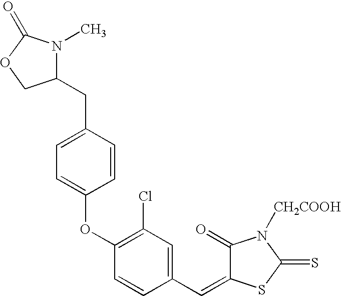 | Yield: 1.30 g (86.66%, ¹HNMR DMSO-d₆ 400 MHz ): δ 2.75 (m, 1H), 2.9 (s, 3H), 3.1 (dd, 1H), 3.94 (m, 1H), 4.0 (t, 1H), 4.2 (t, 1H), 4.75 (s, 2H), 7.0 (m, 3H), 7.2 (d, 2H), 7.3 (m, 2H), 7.6 (s, 1H); m/z^{M+1}: 519.1 |
| 42 | 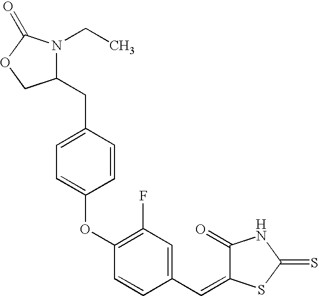 | Yield: 0.63 g (78.7%, ¹HNMR CDCl₃-400 MHz): δ 1.20 (t, 3H), 2.35 (m, 1H), 2.71 (m, 1H), 3.12 (m, 1H), 3.62 (m, 1H), 4.00 (q, 2H), 4.19 (m, 1H), 7.04 (m, 3H), 7.19 (m, 3H), 7.26 (m, 2H), 7.59 (s, 1H), 7.68 (s, 1H); m/z^{M+1}: 459.1 |
| 43 | 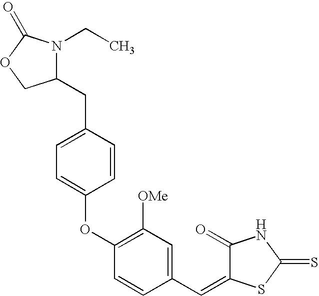 | Yield: 0.82 g (62.12%, ¹HNMR CDCl₃-400 MHz): δ 1.20 (t, 3H), 2.70 (m, 1H), 3.14 (m, 2H), 3.62 (m, 1H), 3.99 (s, 3H), 4.02 (q, 2H), 4.20 (s, 1H), 6.81 (d, 1H), 6.98 (d, 2H), 7.02 (d, 2H), 7.16 (d, 2H), 7.62 (s, 1H); m/z^{M+1}: 471.1 |
| 44 | 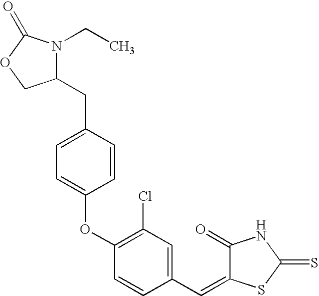 | Yield: 0.61 g (92.4%, ¹HNMR CDCl₃-400 MHz): δ 1.23 (t, 3H), 2.35 (m, 1H), 2.71 (m, 1H), 3.15 (m, 2H), 3.61 (m, 1H), 4.00 (q, 2H), 4.19 (t, 1H), 6.95 (m, 1H), 7.02 (m, 2H), 7.21 (m, 3H), 7.30 (m, 1H), 7.59 (m, 1H); m/z^{M+1}: 475 |
| 45 | 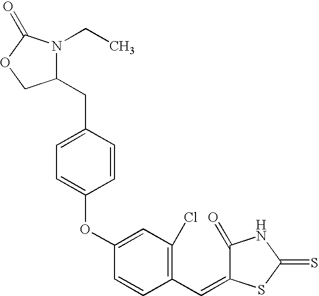 | Yield: 0.60 g (45.1%, ¹HNMR CDCl₃-400 MHz): δ 1.08 (t, 3H), 2.90 (q, 1H), 3.10 (m, 2H), 3.39 (m, 1H), 3.98 (dd, 1H), 4.17 (m, 2H), 4.19 (t, 1H), 7.05-7.21 (m, 5H), 7.38 (d, 2H), 7.53 (d, 1H), 7.72 (s, 1H); m/z^{M+1}: 475 |
| 46 | 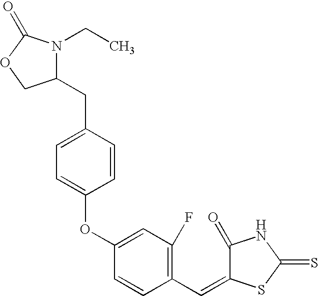 | Yield: 0.60 g (60.0%, ¹HNMR CDCl₃-400 MHz): δ 1.20 (t, 3H), 2.73 (m, 1H), 3.16 (m, 2H), 3.62 (m, 1H), 4.03 (m, qH), 4.22 (m, 1H), 4.19 (t, 1H), 6.72 (m, 1H), 6.85 (m, 1H), 7.02 (m, 2H), 7.23 (m, 2H) 7.36 (m, (m, 1H), 7.82 (m, 1H); m/z^{M+1}: 458.9 |
| 47 | 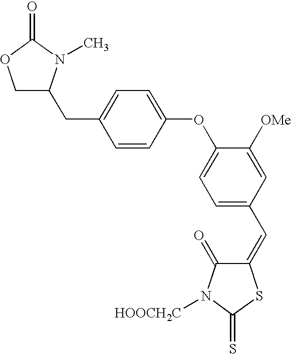 | Yield: 1.19 g (79.33%, ¹HNMR CDCl₃-400 MHz): δ 2.74 (m, 1H), 2.9 (s, 3H), 3.1 (dd, 1H), 3.92 (s, 3H), 3.95 (m, 1H), 3.99 (t, 1H), 4.23 (t, 1H), 4.74 (s, 2H), 6.90 (m, 1H), 6.96 (d, 2H), 7.03 (m, 2H), 7.15 (d, 2H) 7.67 (s, 1H); m/z^{M+1}: 515.1 |
| 48 | 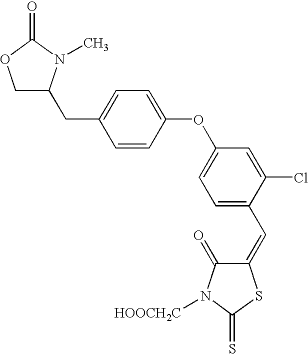 | Yield: 1.5 g (66.6%, ¹HNMR DMSO-d₆- 400 MHz): δ 2.81 (s, 3H), 2.83 (m, 1H), 3.06 (dd, 1H), 3.93 (m, 2H), 4.19 (t, 1H), 4.72 (s, 2H), 7.06 (dd, 1H), 7.16 (m, 2H), 7.25 (d, 1H), 7.39 (d, 2H) 7.62 (d, 1H), 7.93 (s, 1H); m/z^{M+1}: 519.1 |
| 49 | 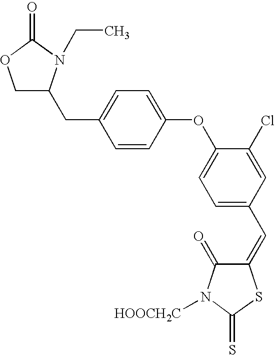 | Yield: 0.820 g (66.6%, ¹HNMR DMSO-d₆- 400 MHz): δ 1.21 (t, 3H), 2.71 (m, 1H), 3.09 (m, 1H), 3.17 (m, 2H), 3.61 (m, 1H), 4.00 (q, 2H), 4.19 (t, 1H), 4.81 (s, 2H), 6.94 (d, 1H), 7.01 (d, 2H), 7.18 (d, 2H), 7.32 (dd, 2H) 7.62 (d, 1H), 7.65 (s, 1H); m/z^{M+1}: 533 |
| 50 | 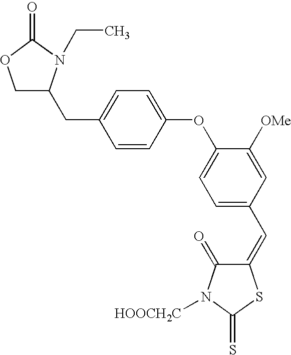 | Yield: 1.2 g (54.05%, ¹HNMR CDCl₃-400 MHz): δ 1.20 (1, 3H), 2.17 (s, 3H), 2.70 (m, 1H), 3.08 (dd, 1H), 3.16 (m, 1H), 3.94 (s, 3H), 3.99 (q, 2H), 4.18 (t, 1H), 4.90 (s, 2H), 6.92 (d, 1H), 7.00 (d, 2H), 7.07 (m, 2H), 7.14 (d, 2H) 7.74 (s, 1H); m/z^{M+1}: 529.1 |
| 51 | 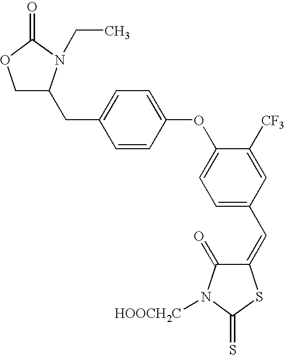 | Yield: 1.2 g (85.7%, ¹HNMR DMSO-d₆- 400 MHz): δ 1.07 (t, 3H), 2.80 (m, 1H), 3.07 (dd, 1H), 3.16 (m, 1H), 3.37 (m, 1H), 3.97 (m, 1H), 4.99 (q, 2H), 4.18 (m, 2H), 4.66 (s, 2H), 7.05 (d, 1H), 7.12 (d, 2H), 7.39 (d, 2H), 7.85 (dd, 2H) 7.95 (s, 1H), 8.13 (d, 1H); m/z^{M+1}: 567.2 |
| 52 | 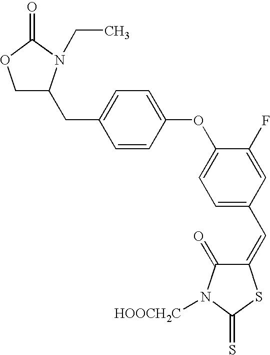 | Yield: 1.19 g (91.6%, ¹HNMR CDCl₃-400 MHz): δ 1.08 (t, 3H), 2.74 (q, 1H), 3.10 (dd, 1H), 3.18 ( m, 1H), 3.59 (m, 1H), 4.0 (q, 2H), 4.17 (t, 1H), 4.85 (s, 2H), 7.05 (m, 3H), 7.18 (d, 2H), 7.24 (m, 1H), 7.29 (dd, 1H), 7.68 (s, 1H); m/z^{M+1}: 534.2 |
| 53 | 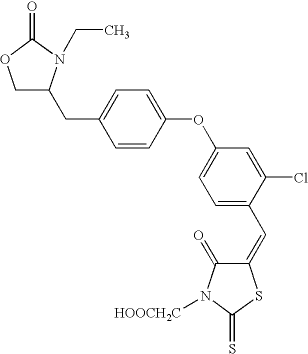 | Yield: 0.85 g (59.4%, ¹HNMR DMSO d₆- 400 MHz): δ 1.08 (t, 3H), 2.77 (m, 1H), 3.10 (m, 2H), 3.33 (m, 1H), 3.99 (m, 1H), 4.19 (q, 2H), 4.72 (s, 2H), 7.06 (d, 1H), 7.14 (d, 2H), 7.25 (s, 1H), 7.39 (d, 2H), 7.62 (d, 1H), 7.93 (s, 1H); m/z^{M+1}: 534.1 |
| 54 | 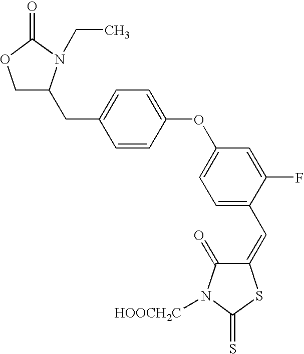 | Yield: 1.1 g (73.1%, ¹HNMR DMSO d₆- 400 MHz): δ 1.09 (t, 3H), 2.73 (m, 1H), 3.10 (m, 2H), 3.18 (m, 1H), 3.39 (m, 1H), 3.98 (m, 1H), 4.19 (m, 2H), 4.75 (s, 2H), 6.99 (m, 2H) 7.14 (m, 2H), 7.39 (d, 2H), 7.60 (t, 1H), 7.79 (s, 1H); m/z^{M+1}: 534.2 |
| 55 | 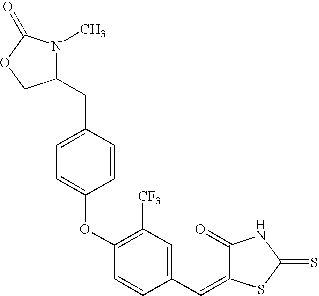 | Yield: 0.510 g (19.6%, ¹HNMR CDCl₃-400 MHz): δ 2.79 (m, 4H), 3.07 (dd, 1H), 3.95 (m, 2H), 4.23 (t, 1H), 7.05 (d, 1H), 7.11 (d, 2H), 7.37 (d, 2H), 7.69 (s, 1H), 7.77 (d, 1H), 8.09 (s, 1H); m/z^{M+1}: 495.1 |
| 56 | 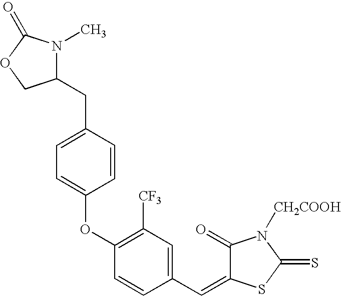 | Yield: 2.75 g (94.5%, ¹HNMR DMSO-d₆- 400 MHz): δ 2.78 (m, 4H), 2.99 (dd, 1H), 3.97 (m, 2H), 4.2 (t, 1H), 4.74 (s, 2H), 7.06 (d, 1H), 7.12 (d, 2H), 7.38 (d, 2H), 7.84 (d, 1H), 7.98 (s, 1H), 8.15 (s, 1H); m/z^{M+1}: 553.1 |
| 57 | 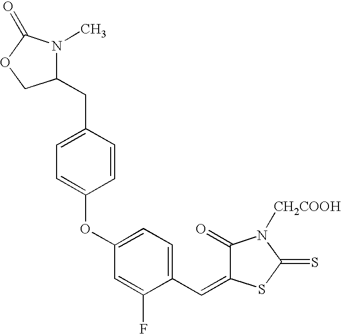 | Yield: 1.10 g (72.36%, ¹HNMR CDCl₃-400 MHz): δ 2.77 (m, 1H), 2.90 (s, 3H), 3.15 (dd, 1H), 3.95 (m, 1H), 4.03 (m, 1H), 4.26 (t, 1H), 4.85 (s, 2H), 6.85 (d, 1H), 7.00 (m, 3H), 7.20 (m, 2H), 7.4 (t, 1H) 7.94 (s, 1H); m/z^{M+1}: 503.2 |
| 58 | 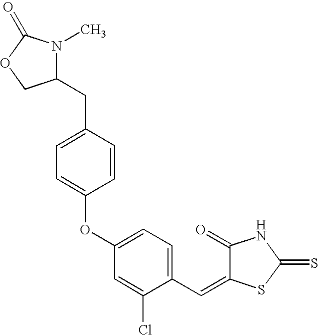 | Yield: 1.49 g (93.7%, ¹HNMR CDCl₃-400 MHz): δ 2.78 (m, 1H), 2.93 (s, 3H), 3.12 (dd, 1H), 3.96 (m, 1H), 4.00 (m, 1H), 4.26 (t, 1H), 6.92 (d, 1H), 7.04 (m, 3H), 7.21 (m, 2H), 7.4 (d, 1H), 7.99 (s, 1H); m/z^{M+1}: 460, 462 |
| 59 | 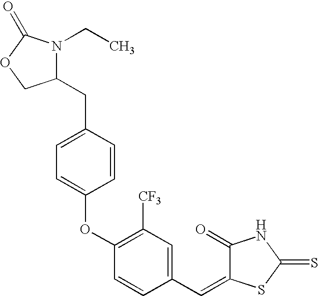 | Yield: 0.950 g (79.1%, ¹HNMR DMSO d₆- 400 MHz): δ 1.07 (t, 3H), 2.77 (m, 1H), 3.07 (m, 2H), 3.39 (m, 1H), 3.98 (m, 1H), 4.16 (m, 2H), 7.04 (d, 1H), 7.11 (d, 2H), 7.25 (d, 2H), 7.74 (s, 1H), 7.78 (d, 1H), 8.09 (s, 1H); m/z^{M+1}: 509.1 |
| 60 | 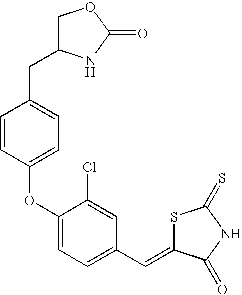 | Yield: 1.20 g (61.7%, ¹HNMR CDCl₃-400 MHz): 2.88 (d, 2H), 4.13 (m, 2H), 4.47 (t, 1H), 6.96 (d, 1H), 7.01 (d, 2H), 7.21 (dd, 2H), 7.31 (m, 1H), 7.50 (s, 1H), 7.58 (d, 1H); m/z^{M+1}: 447 |

### Example 61

### 5-(4-{4-[(3-methyl-2-oxo-1,3-oxazolidin-5-yl)methyl]phenoxy}benzyl)-1,3-thiazolidine-2,4-dione

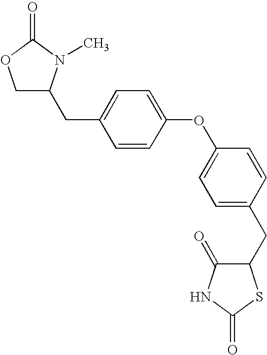

To the solution of (5E)-5-(4-{4-[(3-methyl-2-oxo-1,3-oxazolidin-4-yl)methyl]phenoxy}benzylidene)-1,3-thiazolidine-2,4-dione (0.5 g, 1.21 mmol) in methanol (100 ml) was added 10% Pd/C (0.100 g). The reaction mixture was charged to hydrogenator flask and hydrogenated at 150 psi pressure for 24 hr. The progress of reaction was monitored by HPLC. Solvent was evaporated under reduced pressure to afford off white solid. Yield: 0.415 g (82.66%), ¹HNMR CDCl₃ 400MHz: δ 2.7 (m, 1H), 2.9 (s, 3H), 3.1 (m, 2H), 3.47 (m, 1H), 3.9 (m, 1H), 3.98 (m, 1H), 4.20 (q, 1H), 4.5 (m, 1H), 6.95 (t, 4H), 7.1 (d, 2H), 7.2 (d, 2H) m/z^{M+1}: 412.9

### The Following Compounds are Prepared by the Procedure Given in Example 61

| Ex- | | |
|---|---|---|
| am- | | Analyt- |
| ple | Structure | ical data |
| 62 | 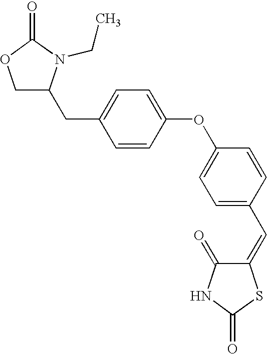 | Yield: 0.35 g (87.28%, ¹HNMR CDCl3 400 MHz): δ 1.22 (t, 3H), 2.64 (m, 1H), 3.15 (m, 3H), 3.47 (dd, 1H), 3.6 (m, 1H), 4.00 (q, 2H), 4.19 (m, 1H), 4.53 (dd, 1H), 6.95 (t, 4H), 7.14 (d, 2H), 7.2 (d, 2H),; m/z^{M+1}: 427.4 |
| 63 | 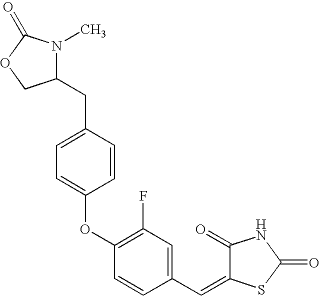 | Yield: 0.130 g (43.18%, ¹HNMR CDCl₃ 400 MHz): δ 2.7 (m, 1H), 2.9 (s, 3H), 3.07 (dd, 1H), 3.1 (m, 1H), 3.46 (dd, 1H), 3.9 (m, 1H), 3.96 (t, 1H), 4.19 (t, 1H), 4.53 (dd, 1H), 6.97 (m, 4H), 7.13 (m, 3H), 8.41 (bs, 1H); m/z^{M+1}: 431.4. |
| 64 | 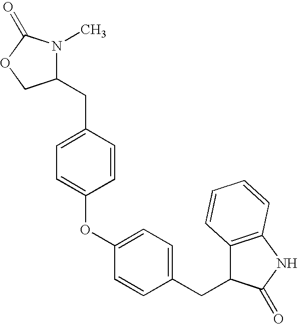 | Yield: 0.24 g (80.0%, ¹HNMR CDCl₃, 400 MHz): δ 2.7 (m, 1H), 2.9 (s, 3H), 3.02 (m, 1H), 3.1 (dd, 1H), 3.42 (dd, 1H), 3.72 (m, 1H), 3.9 (m, 1H), 3.99 (t, 1H), 4.12 (t, 1H), 6.78 (d, 1H), 6.92 (m, 6H), 7.17 (m, 5H), 7.4 (bs, 1H); m/z^{M+1}: 429.4 |
| 65 | 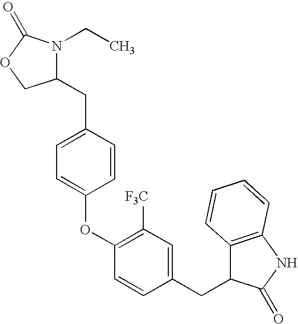 | Yield: 0.42 g (83.8%, ¹HNMR, CDCl₃, 400 MHz): δ 1.20 (t, 3H), 2.6 (q, 1H), 2.88 (d, 1H), 3.13 (m, 3H), 3.60 (dd, 1H), 3.74 (m, 1H), 4.0 (t, 1H), 4.18 (m, 2H), 4.5 (t, 1H) 6.81 (t, 2H), 6.96 (m, 4H), 7.13 (d, 2H), 7.21 (t, 2H), 7.40 (s, 1H), 7.72 (s, 1H); m/z^{M+1}: 511.2 |
| 66 | 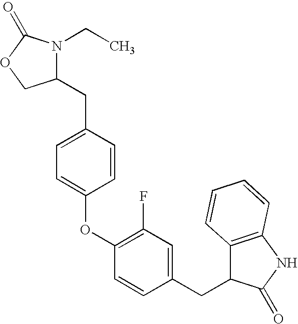 | Yield: 0.25 g (71.4%, ¹HNMR, CDCl₃, 400 MHz): δ 1.19 (t, 3H), 2.56 (q, 1H), 3.16 (m, 1H), 3.39 (dd, 2H), 3.58 (m, 2H), 3.74 (m, 1H), 4.0 (m, 2H), 4.17 (t, 1H), 6.81 (d, 1H), 6.94 (m, 7H), 7.09 (d, 2H), 7.18 (t, 1H), 7.57 (s, 1H); m/z^{M+1}: 461.2 |
| 67 | 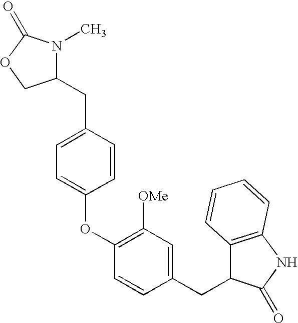 | Yield: 0.18 g (60.0%, ¹HNMR, CDCl₃, 400 MHz): 2.69 (m, 1H), 2.88 (s, 3H), 3.05 (m, 2H), 3.38 (dd, 1H), 3.74 (s, 3H), 3.96 (m, 1H), 3.98 (m, 1H), 6.71 (m, 3H), 6.85 (m, 3H), 6.94 (m, 2H), 7.01 (d, 2H), 7.18 (t, 1H), 7.39 (s, 1H); m/z^{M+1}: 459.3 |
| 68 | 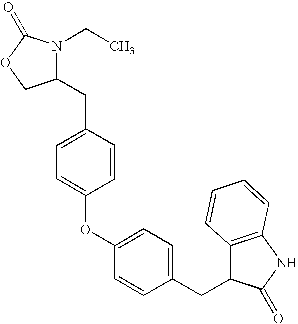 | Yield: 0.16 g (31.90%, ¹HNMR, CDCl₃, 400 MHz): 1.20 (t, 3H), 2.66 (m, 1H), 3.02 (m, 1H), 3.11 (dd, 2H), 3.15 (q, 1H), 3.42 (dd, 1H), 3.60 (q, 1H), 3.70 (m, 1H), 4.00 (q, 2H), 4.18 (m,1H), 6.80-6.89 (m, 7H), 7.1 (m, 4H), 7.20 (m, 1H), 7.70 (s, 1H); m/z^{M+1}: 443.2 |
| 69 | 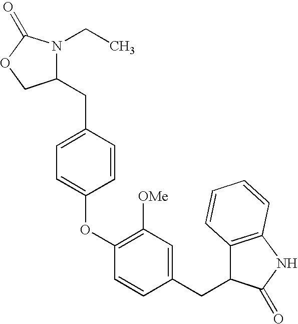 | Yield: 0.22 g (68.0%, ¹HNMR, CDCl₃, 400 MHz): 1.20 (t, 3H), 2.64 (q, 1H), 3.14 (m, 1H), 3.31 (m, 1H), 3.44 (dd, 1H), 3.69 (s, 3H), 3.74 (dd, 1H), 3.98 (q, 2H), 4.16 (m, 1H), 6.73 (m, 2H), 6.82 (m, 4H), 6.94 (m, 2H), 7.05 (d, 2H), 7.18 (t, 1H), 7.49 (s, 1H); m/z^{M+1}: 473.2 |
| 70 | 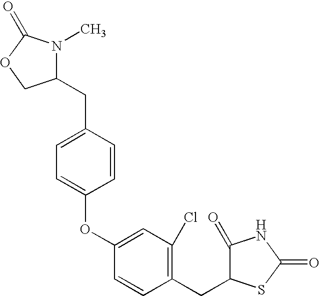 | Yield: 0.120 g (60.0%, ¹HNMR CDCl₃- 400 MHz): 2.88 (q, 1H), 2.96 (s, 3H), 3.12 (m, 2H), 3.85 (m, 1H), 3.92 (m, 1H), 4.00 (m, 1H), 4.60 (m, 1H), 5.14 (m, 1H), 6.85 (dd, 1H), 7.01 (m, 3H), 7.2 (m, 2H); m/z^{M+1}: 447.1 |
| 71 | 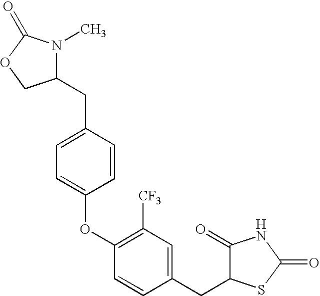 | Yield: 0.250 g (49.8%, ¹HNMR DMSO d₆-400 MHz): 2.77 (m, 4H), 3.04 (dd, 1H), 3.24 (m, 1H), 3.85 (m, 1H), 3.95 (m, 2H), 4.19 (m, 1H), 4.97 (dd, 1H), 6.97 (m, 3H), 7.31 (d, 2H), 7.53 (d, 1H), 7.67 (m, 1H); m/z^{M+1}: 481.1 |
| 72 | 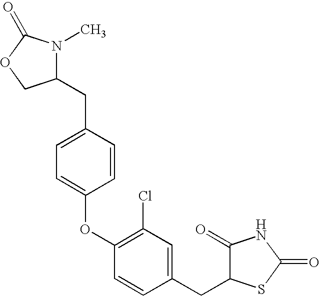 | Yield: 0.480 g (86.9%, ¹HNMR CDCl₃- 400 MHz): 2.7 (m, 1H), 2.9 (s, 3H), 3.08 (m, 2H), 3.45 (m, 1H), 3.93 (m, 1H), 3.94 (t, 1H), 4.23 (t, 1H), 4.49 (s, 1H), 6.91 (d, 3H), 7.06 (m, 2H), 7.12 (m, 3H), 7.35 (s, 1H); m/z^{M+1}: 447.1 |
| 73 | 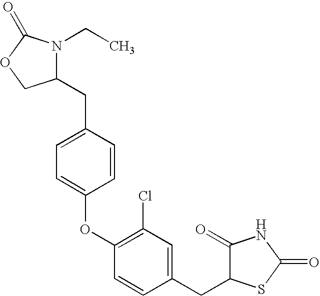 | Yield: 0.130 g (25.8%, ¹HNMR CDCl₃- 400 MHz): 1.19 (t, 3H), 2.7 (m, 1H), 3.10 (m, 3H), 3.49 (m, 1H), 3.59 (m, 1H), 3.98 (q, 2H), 4.18 (t, 1H), 4.55 (m, 1H), 6.91 (d, 3H), 7.06 (m, 3H), 7.52 (s, 1H), 7.98 (bs, 1H); m/z^{M+1}: 460.9 |
| 74 | 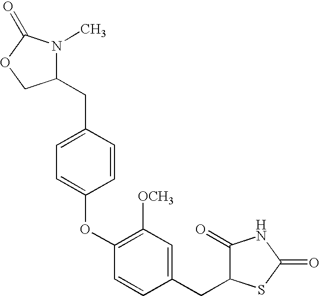 | Yield: 0.185 g (92.5%, ¹HNMR CDCl₃- 400 MHz): 2.71 (m, 1H), 2.89 (s, 3H), 3.06 (m, 1H), 3.15 (m, 1H), 3.54 (m, 1H), 3.89 (s, 3H), 3.90 (m, 1H), 3.98 (q, 1H), 4.23 (t, 1H), 4.55 (dd, 1H), 6.78 (m, 1H), 6.87 (m, 4H), 7.09 (d, 2H); m/z^{M+1}: 460.2 |
| 75 | 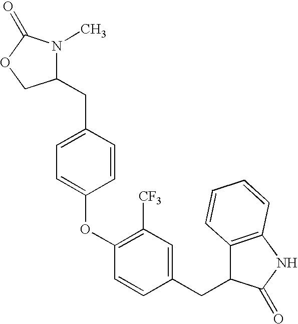 | Yield: 0.108 g (90.0%, ¹HNMR, DMSO- d₆-400 MHz): δ 2.74 (m, 4H), 2.99 (dd, 1H), 3.16 (m, 1H), 3.31 (dd, 1H), 3.87 (t, 1H), 3.96 (m, 2H), 4.20 (t, 1H), 4.02 (t, 1H), 4.26 (t, 1H), 6.75 (d, 2H) 6.80 (d, 3H) 6.92 (m, 2H), 7.08 (m, 2H), 7.27 (m, 3H), 7.43 (s, 1H); m/z^{M+1}: 497.3 |

### Example 76

### 5-(4-{4-[(3-methyl-2-oxo-1,3-oxazolidin-4-yl)methyl]phenoxy}benzyl)-2-thioxo-1,3-thiazolidin-3-yl]acetic acid

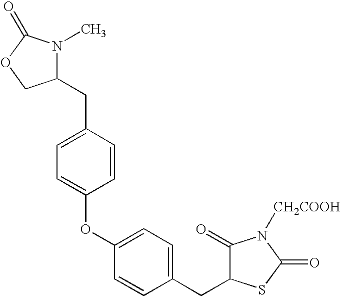

To the solution of 3-methyl-5-(4-{4-[(E)-(3-aceticacid-4-oxo-2-thioxo-1,3-thiazolidin-5-ylidene)methyl]phenoxy}benzyl)-1,3-oxazolidin-2-one (0.5 g, 1.03 mmol) in toluene (30 ml) was added 1,4-dihydro-3,5-dicarbethoxy-2,6-dimethyl pyridine (0.68 g, 2.68 mmol) and silica gel 60-120 (1.5 g). The reaction mixture was stirred for 24 hr. at 80-85° C. The progress of reaction was monitored by HPLC. Solvent was evaporated under reduced pressure to yield crude product which was purified by column cromatography. Yield 0.18 g (36%, ¹HNMR CDCl3 400 MHz): δ 2.74 (m, 1H), 2.9 (s, 3H), 3.1 (m, 2H), 3.53 (m, 1H), 3.9 (m, 1H), 4.02 (m, 1H), 4.09 (m, 1H), 4.23 (m, 1H), 4.66 (s, 2H), 6.94 (m, 4H), 7.11 (dd, 2H), 7.19 (dd, 2H); m/z^{M+1}: 487.2

### The Following Compound are Prepared by the Procedure Give in Example 76

| Example | Structure | Analytical data |
|---|---|---|
| 77 | 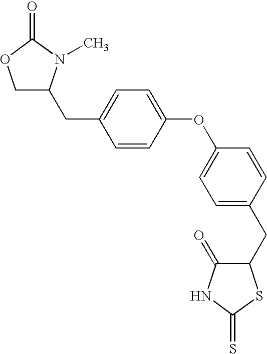 | Yield: 0.24 g (47.8%, ¹HNMR CDCl₃ 400 MHz): δ 2.7 (m, 1H), 2.9 (s, 3H), 3.1 (dd, 1H), 3.2 (m, 1H), 3.47 (dd, 1H), 3.9 (m, 1H), 4.0 (t, 1H), 4.25 (t, 1H), 4.6 (dd, 1H), 6.96 (dd, 4H), 7.1 (d, 2H), 7.2 (d, 2H); m/z^{M+1}: 429.1 |
| 78 | 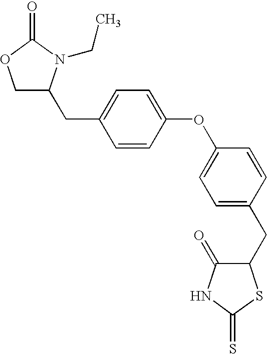 | Yield: 0.26 g (51.79%, ¹HNMR CDCl₃ 400 MHz): δ 1.22 (1, 3H), 2.68 (m, 1H), 3.15 (m, 3H), 3.46 (dd, 1H), 3.6 (m, 1H), 4.0 (q, 2H), 4.19 (m, 1H), 4.6 (dd, 1H), 6.96 (m, 4H), 7.12 (d, 2H), 7.2 (d, 1H), 9.0 (bs, 1H); m/z^{M+1}: 443.4 |
| 79 | 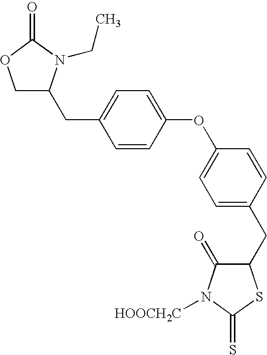 | Yield: 0.08 g (20%, ¹HNMR CDCl₃ 400 MHz): δ 1.19 (t, 3H), 2.67 (m, 2H), 3.05 (m, 3H), 3.59 (m, 2H), 4.0 (q, 2H), 4.17 (m, 1H), 4.59 (bs, 2H), 6.92 (t, 4H), 7.09 (d, 2H), 7.15 (d, 2H); m/z^{M+1}: 501.1 |
| 80 | 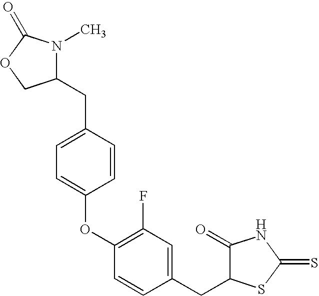 | Yield: 0.21 g (46.6%, ¹HNMR CDCl₃ 400 MHz): δ 2.7 (m, 1H), 2.9 (s, 3H), 3.1 (dd, 1H), 3.22 (m, 1H), 3.45 (dd, 1H), 3.9 (m, 1H), 3.97 (t, 1H), 4.21 (t, 1H), 4.61 (dd, 1H), 6.93 (d, 2H), 6.99 (m, 2H), 7.09 (m, 3H), 8.9 (bs, 1H) m/z^{M+1}: 447 |
| 81 | 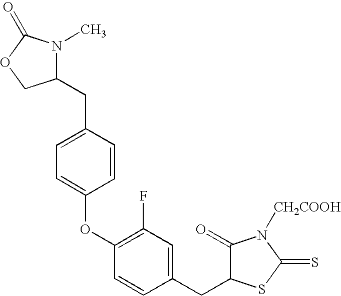 | Yield: 0.09 g (15%, ¹HNMR CDCl₃ 400 MHz): δ 2.8 (m, 1H), 2.9 (s, 3H), 2.95 (m, 1H), 3.1 (m, 1H), 3.4 (m, 1H), 3.9 (m, 1H), 4.0 (t, 1H), 4.23 (t, 1H), 4.56 (m, 1H), 4.7 (s, 2H), 6.89 (m, 4H), 7.06 (m, 3H); m/z^{M+1}: 505.3 |
| 82 | 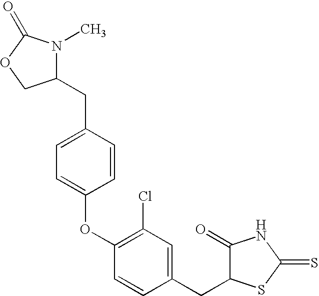 | Yield: 0.13 g (21.6%, ¹HNMR CDCl₃ 400 MHz); δ 2.7 (m, 1H), 2.9 (s, 3H), 3.1 (dd, 1H), 3.19 (m, 1H), 3.45 (m, 1H), 3.9 (m, 1H), 3.97 (t, 1H), 4.24 (t, 1H), 4.61 (dd, 1H), 6.92 (m, 3H), 7.1 (m, 3H), 7.34 (s, 1H); m/z^{M+1}: 463.1 |
| 83 | 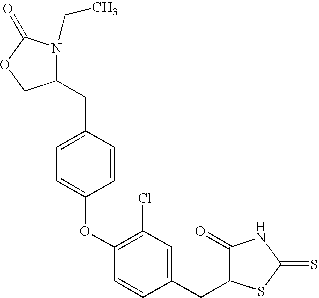 | Yield: 0.057 g (11.0%, ¹HNMR CDCl₃-400 MHz): δ 1.21 (t, 3H), 2.70 (m, 1H), 3.14 (m, 3H), 3.47 (m, 1H), 3.60 (m, 3H), 3.99 (q, 2H), 4.18 (m, 1H), 6.92 (m, 3H), 7.12 (m, 3H), 7.35 (s, 1H); m/z^{M+1}: 477, 478.7 |
| 84 | 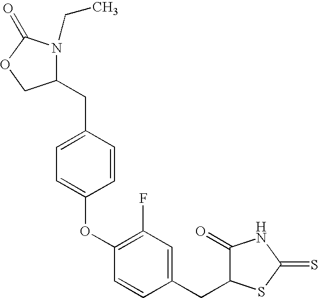 | Yield: 0.09 g (22.5%, ¹HNMR CDCl₃-400 MHz): δ 1.21 (t, 3H), 2.70 (m, 1H), 3.14 (m, 3H), 3.47 (m, 1H), 3.58 (m, 1H), 3.99 (q, 2H), 4.20 (m, 1H), 4.58 (dd, 1H), 6.92 (m, 2H), 7.02 (m, 2H), 7.10 (m, 3H); m/z^{M+1}: 461.2 |
| 85 | 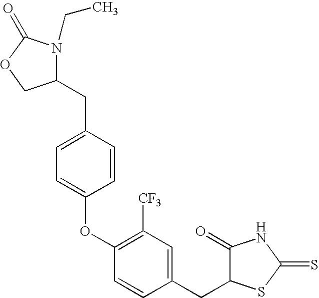 | Yield: 0.180 g (29.9%, ¹HNMR DMSO-d₆ 400 MHz): δ 1.07 (t, 3H), 2.70 (m, 1H), 3.12 (m, 2H), 3.34 (m, 1H), 3.40 (q, 2H), 3.95 (m, 1H), 4.0 (m, 1H), 4.18 (m, 1H), 6.94 (m, 3H), 7.32 (d, 2H), 7.5 (d, 2H), 7.66 (s, 1H); m/z^{M+1}: 511.1 |
| 86 | 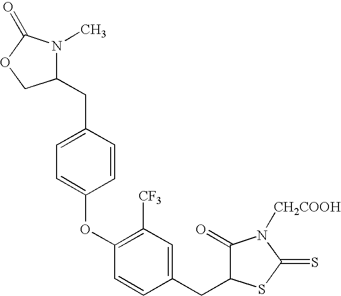 | Yield: 0.300 g (53.3%, ¹HNMR, DMSO d₆- 400 MHz): δ 2.79 (m, 4H), 3.16 (d, 1H), 3.28 (d, 1H), 3.47 (d, 1H), 3.96 (m, 3H), 4.21 (m, 1H), 4.41 (s, 2H), 6.9 (m, 3H) 7.32 (d, 2H), 7.52 (d, 1H), 7.68 (s, 1H); m/z^{M+1}: 555.1 |
| 87 | 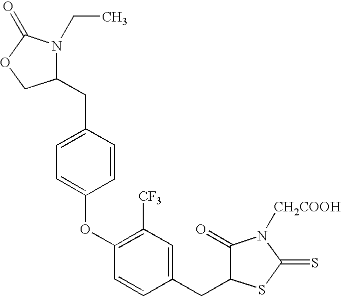 | Yield: 0.140 g (20.5%, ¹HNMR, DMSO d₆- 400 MHz): δ 1.08 (t, 3H), 2.72 (m, 1H), 3.12 (m, 2H), 3.38 (m, 2H), 3.45 (d, 1H), 3.68 (m, 1H), 3.97 (m, 1H), 4.15 (m, 1H), 4.17 (q, 2H), 4.54 (s, 2H), 5.21 (m, 1H), 6.95 (m, 3H) 7.32 (d, 2H), 7.52 (d, 1H), 7.71 (s, 1H); m/z^{M+1}: 569.1 |
| 88 | 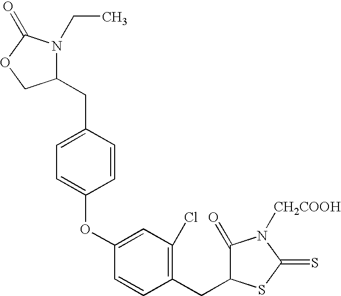 | Yield: 0.170 g (20.5%, ¹HNMR, DMSO d₆- 400 MHz): δ 1.06 (t, 3H), 2.73 (m, 1H), 3.12-3.38 ( m, 4H), 3.54 (dd, 1H), 3.97 (m, 1H), 4.15 (m, 2H), 4.29 (s, 2H), 4.95 (m, 1H), 6.91 (m, 1H) 7.01 (m, 3H), 7.34 (d, 2H), 7.38 (d, 1H); m/z^{M+1}: 535 |
| 89 | 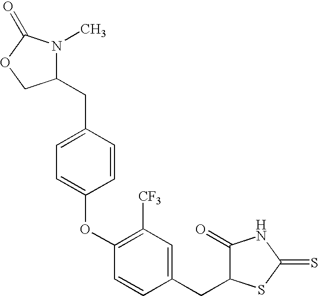 | Yield: 0.125 g (31.1%, ¹HNMR CDCl₃-400 MHz): δ 2.76 (m, 1H), 2.91 (s, 3H), 3.09 (dd, 1H), 3.24 (m, 1H), 3.49 (m, 1H), 3.92 (m, 1H), 4.00 (t, 1H), 4.21 (t, 1H), 4.61 (dd, 1H), 6.88 (d, 1H), 6.98 (d, 2H), 7.15 (d, 2H), 7.34 (dd, 1H), 7.53 (d, 1H); m/z^{M+1}: 497.1 |
| 90 | 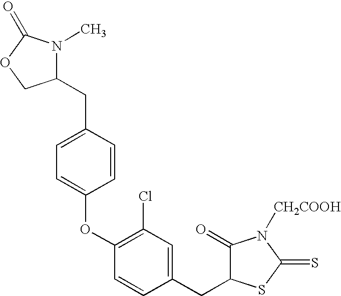 | Yield: 0.364 g (60.46%, ¹HNMR CDCl₃-400 MHz): δ 2.73 (m, 1H), 2.89 (s, 3H), 3.06 (m, 2H), 3.52 (m, 1H), 3.99 (m, 2H), 4.19 (t, 1H), 4.60 (m, 2H), 6.90 (m, 3H), 7.1 (m, 3H), 7.27 (s, 1H); m/z^{M+1}: 521.1 |
| 91 | 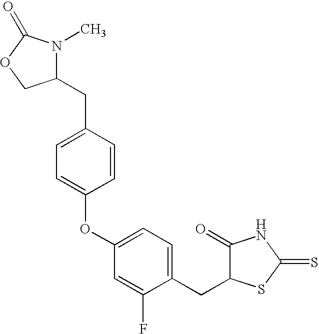 | Yield: 0.075 g (37.5%, ¹HNMR CDCl₃-400 MHz): δ 2.73 (m, 1H), 2.92 (s, 3H), 3.12 (dd, 1H), 3.66 (dd, 1H), 3.92 (m, 1H), 4.00 (m, 1H), 4.24 (t, 1H), 4.67 (m, 1H), 6.68 (m, 2H), 6.99 (d, 2H), 7.15 (m, 3H); m/z^{M+1}: 447.1 |
| 92 | 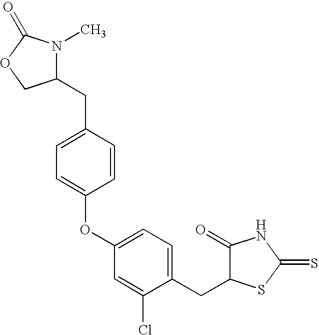 | Yield: 0.370 g (74.0%, ¹HNMR CDCl₃-400 MHz): δ 2.76 (m, 1H), 2.92 (s, 3H), 3.09 (m, 2H), 3.75 (dd, 1H), 3.92 (m, 1H), 4.00 (m, 1H), 4.24 (t, 1H), 4.75 (m, 1H), 6.84 (dd, 1H), 6.99 (m, 3H), 7.16 (m, 3H); m/z^{M+1}: 463.1 |

### Protocols for Bilogy Testing

### Glucose Uptake Assay Using 3T3-L1 Cells

3T3-L1 cells were differentiated by the addition of differentiation cocktail (72 μg/ml insulin, 0.5 mM IBMX, 400 ng/ml Dexamethasone) for 4 days and later fed with media without differentation cocktail for 7-8 days. After differentiation the cells were incubated with the either reference compound BLX-1002 or compounds listed in the table 1 at 1 μM concentrations for 72 hours and carried out the glucose uptake assay for 10 min by the addition of KRP buffer supplemented with 2.5 μCi/ml ¹⁴C deoxy glucose. Stimulation Index is defined as the amount of ¹⁴C Deoxyglucose uptake induced by 1 μM of BLX-1002 incubated for 72 hrs in an assay condition as per protocol described above with differentiated 3T3-L1 adipocytes. Values of compounds mentioned in table-1 are with reference to stimulation index of reference compound BLX-1002. Example 78 and few other compounds mentioned in table-1 have good glucose uptake activity. The results are shown in Table-1.

TABLE-1
Effect of compounds on glucose uptake assay in 3T3-L1 cells

| Example No | Stimulation Index |
|---|---|
| BLX-1002 | 1.00 |
| 1 | 0.96 |
| 2 | 0.75 |
| 3 | 1.00 |
| 4 | 0.90 |
| 5 | 0.87 |
| 6 | 0.84 |
| 8 | 1.01 |
| 10 | 0.82 |
| 11 | 1.12 |
| 13 | 0.80 |
| 15 | 0.96 |
| 19 | 1.13 |
| 20 | 0.86 |
| 21 | 0.82 |
| 22 | 1.07 |
| 23 | 0.94 |
| 24 | 0.77 |
| 25 | 1.15 |
| 26 | 0.92 |
| 27 | 0.82 |
| 31 | 0.99 |
| 32 | 0.89 |
| 33 | 0.84 |
| 34 | 0.78 |
| 35 | 0.95 |
| 36 | 0.87 |
| 37 | 0.95 |
| 38 | 0.62 |
| 40 | 1.07 |
| 41 | 1.12 |
| 42 | 0.69 |
| 43 | 0.96 |
| 44 | 1.02 |
| 45 | 0.94 |
| 46 | 0.90 |
| 48 | 0.85 |
| 49 | 0.88 |
| 50 | 0.70 |
| 51 | 0.97 |
| 52 | 0.86 |
| 53 | 0.92 |
| 54 | 0.92 |
| 55 | 0.68 |
| 56 | 0.64 |
| 57 | 0.94 |
| 58 | 0.86 |
| 59 | 0.81 |
| 61 | 0.90 |
| 62 | 0.99 |
| 63 | 0.75 |
| 64 | 1.09 |
| 65 | 0.85 |
| 66 | 0.94 |
| 67 | 1.01 |
| 70 | 1.00 |
| 71 | 1.03 |
| 72 | 1.02 |
| 73 | 1.19 |
| 74 | 0.84 |
| 75 | 0.78 |
| 77 | 0.92 |
| 78 | 1.06 |
| 79 | 0.85 |
| 80 | 0.95 |
| 81 | 0.96 |
| 82 | 1.06 |
| 83 | 0.82 |
| 84 | 0.93 |
| 85 | 1.15 |
| 86 | 1.13 |
| 87 | 1.00 |
| 88 | 0.94 |
| 89 | 0.86 |
| 90 | 0.97 |
| 91 | 0.97 |
| 92 | 0.94 |

### Adipogenesis Assay in 3T3-L1

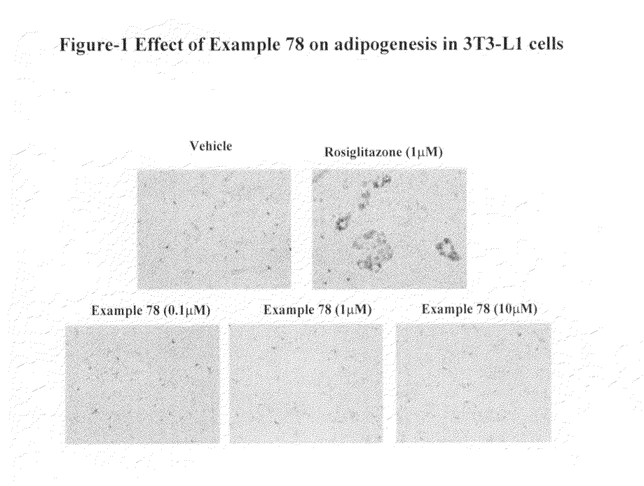

All known PPARγ agonists induce differentiation in fibroblast cells. The adipogenic potential of these compounds is correlated with their affinity to this receptor. To check quickly the affinity of compounds to these receptors, 3T3-L1 fibroblasts were treated with either DMSO control or rosiglitazone as positive control or the compounds at 1 μM concentration for several days. On day 13^{th}, the differentiated adipocytes were stained with Oil-red-O (Sigma) and washed thoroughly to remove unbound stain and visualized under Olympus microscope. PPARγ agonist rosiglitazone strongly induced adipogenesis in this cell system whereas example 78 remained unchanged, further the DRC for example 78 for Adipogenesis was carried out as described above and example 78 did not show significant staining at tested concentrations. This is an indirect proof that example 78 have no affinity to PPARγ receptor. The results are shown in FIG. 1.

### DPP IV Assay

DPP IV assay is carried by using human plasma as a source of DPP IV. The compounds were incubated at a concentration of 1 and 10 μM in assay buffer containing DPP IV enzyme. The compounds were incubated for 1 hr and then the substrate H-gly-pro AMC was added and further incubated for 20 min and then the reaction was stopped on addition of 25% glacial acetic acid. The plates were read in a spectrofluorimeter to get RFU on setting excitation wavelength of 360 nm and emission wavelength of 460 nm. Percentage inhibition is calculated as compared to vehicle control. All compounds studied as shown in table-2 did not produce significant DPP IV inhibition. The results are shown in table-2.

TABLE-2
DPP IV inhibition of compounds

% DPP IV Inhibition

| Example No | 1 μM | 10 μM |
|---|---|---|
| 7 | 3.55 | 7.75 |
| 14 | 4.64 | 7.42 |
| 15 | 7.07 | 9.86 |
| 31 | 4.77 | 7.25 |
| 34 | 9.52 | −0.7 |
| 55 | 1.99 | 9.92 |
| 59 | −1.26 | 12.38 |
| 75 | 2.44 | 7.64 |

### Antidiabetic Activity in Streptozotocin Induced Diabetic Mice

Swiss albino mice of either sex were used in the study at the age of 10 weeks. Diabetes was induced in animals by injecting streptozotocin by i.p. route at a dose of 200 mg/kg body weight. 48 hours after streptozotocin administration, the animals were kept for fasting for 6 hours and blood was collected and plasma separated and glucose was estimated. Animals showing greater than 200 mg/dl glucose levels were considered as diabetic and these animals were randomly distributed into various groups. The compounds listed in the table 3 were administered at a dose of 50 mg/kg body weight by oral route for 7 days. Later animals were fasted for 6 hours and blood was collected and plasma separated. Biochemical estimations like glucose, cholesterol and triglycerides were carried out using the plasma. The effect of compounds mentioned in the table was expressed in terms of percentage reduction in biochemical values as compared to control group. The results are as shown in the table 3.

TABLE-3
Effect of compounds in streptozotocin induced diabetic mice model

| | % Reduction | | |
|---|---|---|---|
| Example No | Glucose | Cholesterol | Triglycerides |
| 3 | NR | NR | 3.8 |
| 34 | 50.9 | NR | 73.4 |
| 37 | 22.5 | NR | NR |
| 40 | 34.2 | NR | 17.8 |
| 41 | 66.5 | NR | NR |
| 62 | 36.2 | NR | 24.7 |
| 71 | 49.9 | NR | NR |
| 78 | 45.4 | 59 | 44.2 |
| 86 | 48.8 | NR | 15.5 |

### NR=No Reduction

TNF-α, IL-6 and IL-1β Inhibition in Human Peripheral Blood Monocytic Cells (hPBMC)

Human PBMC cells were cultured and incubated with compounds listed in table-4 at 1 and 10 μM concentrations. Cells (1×10⁶/mL) were challenged with lipopolysaccharides (LPS) at a concentration of (100 ng/mL) for 20 hours. Cell supernatant was analyzed for the presence of TNF-α, IL-1β and IL-6 cytokines by antibody directed enzyme-linked immunoassay. As shown in table-3, the example 69 can inhibit the production of three major pro-inflammatory cytokines at tested concentrations. No significant change in cell viability was observed with incubation of cells in the presence of highest concentration of the compounds tested. These results strongly indicate that example 69 is effective in reducing the production of pro-inflammatory cytokines. The results are shown in table-4.

TABLE-4
Effect of compounds on cytokine inhibition in Human PBMC assay

| | % Inhibition | | | | | |
|---|---|---|---|---|---|---|
| Example | TNF alpha | | IL-6 | | IL1beta | |
| No | 1 μM | 10 μM | 1 μM | 10 μM | 1 μM | 10 μM |
| 4 | 8.77 | 2.85 | NA | NA | 1.55 | 10.70 |
| 5 | 1.72 | 9.77 | NA | NA | NA | NA |
| 7 | 0.93 | 6.43 | NA | NA | NA | NA |
| 10 | 20.20 | 34.99 | 15.47 | 7.18 | NA | NA |
| 11 | 18.77 | 26.07 | NA | NA | 17.10 | NA |
| 12 | 13.64 | 28.08 | 2.67 | 1.65 | NA | NA |
| 13 | 6.54 | 17.24 | 4.18 | NA | NA | NA |
| 14 | 15.91 | 25.35 | NA | NA | NA | 32.10 |
| 15 | 24.54 | 15.94 | 1.16 | NA | 1.78 | NA |
| 16 | 6.60 | NA | NA | NA | NA | NA |
| 64 | 9.43 | 15.58 | NA | NA | 8.77 | 25.89 |
| 65 | 4.60 | 1.67 | NA | NA | NA | NA |
| 66 | NA | NA | 3.58 | NA | NA | NA |
| 67 | 20.87 | 33.95 | NA | 2.12 | 12.11 | 20.92 |
| 68 | NA | NA | NA | NA | NA | NA |
| 69 | 35.23 | 50.59 | 2.98 | 12.40 | 33.26 | 39.70 |

### Inhibition of Breast Cancer Cell Growth

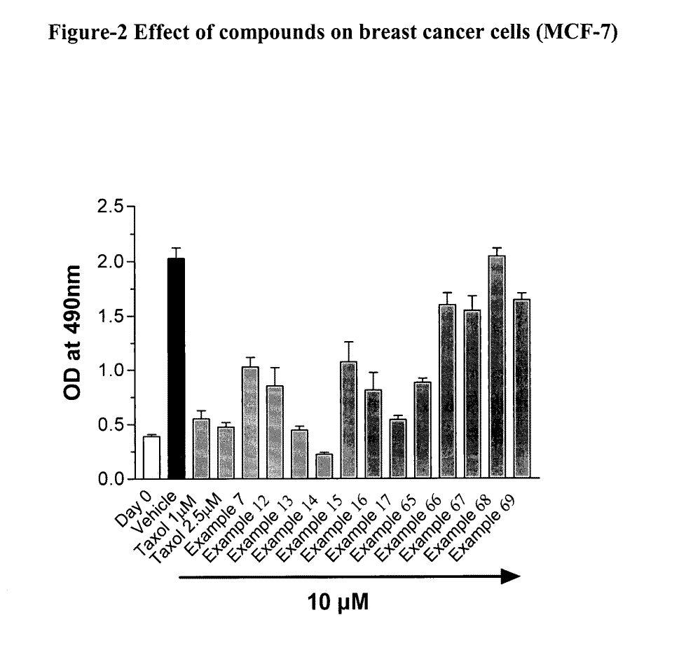

MCF-7 is a breast cancer cell line and they were grown in 96 well plates at 1000 cells/well. The cells were pretreated with compounds mentioned in FIG. 2 at 10 μM concentration or taxol at 1 and 2.5 μM concentration or DMSO for six consecutive days. Every 48 hrs they were stained with MTS dye and viability was checked accordingly. The example 14 is a strong inhibitor of the breast cancer cell growth. The results are shown in FIG. 2.

### Inhibition of Colon Cancer Cell Growth

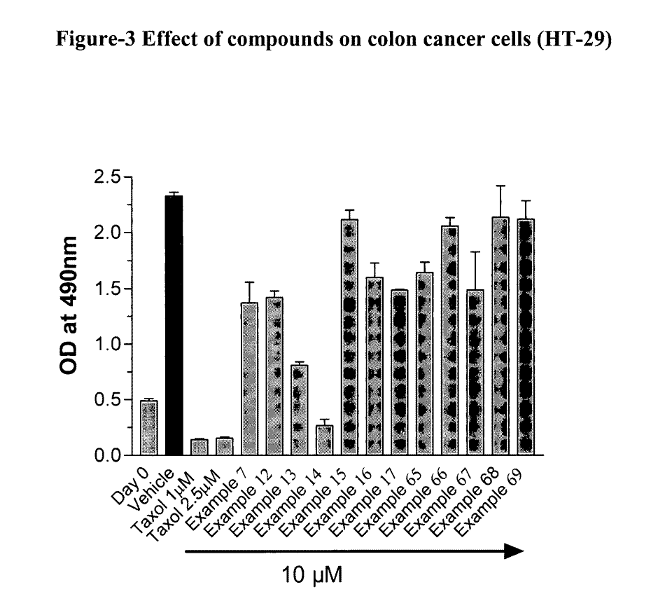

HT-29 is a colon cancer cell line and they were grown in 96 well plates with seeding concentration of 1000 cells/well. The cells were pretreated with compounds mentioned in FIG. 3 at 10 μM concentrations or taxol at 1 and 2.5 μM concentration or DMSO for six consecutive days. Every 48 hrs they were stained with MTS dye and viability was checked accordingly. The example 14 is a strong inhibitor of the breast cancer cell growth. The results are shown in FIG. 3.

We claim:

1. Novel Heterocyclic derivatives of formula (I) 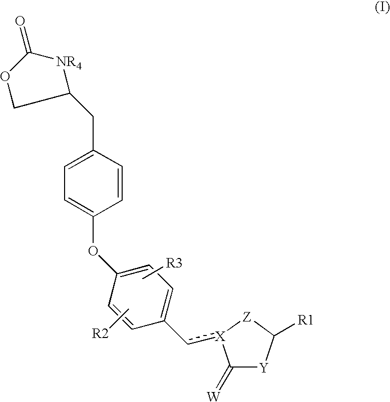
their derivatives, their analogs, their tautomeric forms, their stereoisomers, their polymorphs, their hydrates, their solvates, their pharmaceutically acceptable salts, their pharmaceutical compositions, and their prodrugs thereof, wherein [symbol] represents an optional bond;
W represents O or S;
X represents C, CH or N;
Y represents NR₅, S or O, wherein R₅ represents hydrogen, substituted or unsubstituted alkyl, alkenyl, —CH₂COOR, or aryl, or counter ion; wherein R represents H or alkyl group;
Z represents CR₆ or S; R₁ represents ═O, ═S or together with R₆ forms fused 5 or 6 membered aromatic or heteroaromatic ring system containing carbon atoms or 1 or 2 heteroatoms selected from O, S or N;
R₂, R₃, may be same or different and independently represent hydrogen, halogen, hydroxy, nitro, cyano, formyl, amino, alkyl, haloakyl, alkoxy group;
R₄ may be same or different and independently represent H, COR₇, substituted or unsubstituted groups selected from alkyl, alkenyl, aryl, aryloxy, alkoxy, heteroaryl or heterocyclyl; where R₇ represents H, substituted or unsubstituted groups selected from alkyl, alkenyl, aryl, aryloxy, alkoxy or aralkoxy.

2. Novel Heterocyclic derivatives as claimed in claim 1, wherein the [symbol] represent single bond or no bond.

3. Novel Heterocyclic derivatives as claimed in claim 1, are selected from a group comprising of:
1) 5-(4-{4-[(3-methyl-2-oxo-1,3-oxazolidin-4-yl)methyl]phenoxy}benzylidene)-1,3-thiazolidine-2,4-dione;
2) 5-(4-{4-[(2-oxo-1,3-oxazolidin-4-yl)methyl]phenoxy}benzylidene)-1,3-thiazolidine-2,4-dione;
3) 5-(4-{4-[(3-ethyl-2-oxo-1,3-oxazolidin-4-yl)methyl]phenoxy}benzylidene)-1,3-thiazolidine-2,4-dione;
4) 5-(4-{4-[(3-methyl-2-oxo-1,3-oxazolidin-4-yl)methyl]phenoxybenzylidene)-1,3-dihydro-2H-indol-2-one;
5) 5-(4-{4-[(3-ethyl-2-oxo-1,3-oxazolidin-4-yl)methyl]phenoxybenzylidene)-1,3-dihydro-2H-indol-2-one;
6) 5-(3-fluoro-4-{4-[(3-methyl-2-oxo-1,3-oxazolidin-4-yl)methyl]phenoxy}benzylidene)-1,3-thiazolidine-2,4-dione;
7) 5-(3-fluoro-4-{4-[(3-methyl-2-oxo-1,3-oxazolidin-4-yl)methyl]phenoxybenzylidene)-1,3-dihydro-2H-indol-2-one;
8) 5-(3-chloro-4-{4-[(3-methyl-2-oxo-1,3-oxazolidin-4-yl)methyl]phenoxy}benzylidene)-1,3-thiazolidine-2,4-dione;
9) 5-(3-chloro-4-{4-[(3-methyl-2-oxo-1,3-oxazolidin-4-yl)methyl]phenoxybenzylidene)-1,3-dihydro-2H-indol-2-one;
10) 5-(3-chloro-4-{4-[(3-ethyl-2-oxo-1,3-oxazolidin-4-yl)methyl]phenoxybenzylidene)-1,3-dihydro-2H-indol-2-one;
11) 5-(3-methoxy-4-{4-[(3-methyl-2-oxo-1,3-oxazolidin-4-yl)methyl]phenoxy benzylidene)-1,3-dihydro-2H-indol-2-one;
12) 5-(3-fluoro-4-{4-[(3-ethyl-2-oxo-1,3-oxazolidin-4-yl)methyl]phenoxybenzylidene)-1,3-dihydro-2H-indol-2-one;
13) 3-(4-{3-methoxy-4-[(3-ethyl-2-oxo-1,3-oxazolidin-5-yl)methyl]phenoxy}benzylidene)-1,3-dihydro-2H-indol-2-one;
14) 3-(4-{3-triflouromethyl-4-[(3-methyl-2-oxo-1,3-oxazolidin-5-yl)methyl]phenoxy}benzylidene)-1,3-dihydro-2H-indol-2-one;
15) 3-(4-{3-triflouromethyl-4-[(3-ethyl-2-oxo-1,3-oxazolidin-5-yl)methyl]phenoxy}benzylidene)-1,3-dihydro-2H-indol-2-one;
16) 5-(2-chloro-4-{4-[(3-methyl-2-oxo-1,3-oxazolidin-4-yl)methyl]phenoxybenzylidene)-1,3-dihydro-2H-indol-2-one;
17) 3-(4-{2-chloro-4-[(3-ethyl-2-oxo-1,3-oxazolidin-5-yl)methyl]phenoxy}benzylidene)-1,3-dihydro-2H-indol-2-one
18) 5-(3-trifluoromethyl-4-{4-[(3-methyl-2-oxo-1,3-oxazolidin-4-yl)methyl]phenoxy}benzylidene)-1,3-thiazolidine-2,4-dione;
19) 5-(3-methoxy-4-{4-[(3-methyl-2-oxo-1,3-oxazolidin-4-yl)methyl]phenoxy}benzylidene)-1,3-thiazolidine-2,4-dione;
20) 5-(2-chloro-4-{4-[(3-methyl-2-oxo-1,3-oxazolidin-4-yl)methyl]phenoxy}benzylidene)-1,3-thiazolidine-2,4-dione;
21) 5-(3-chloro-4-{4-[(3-ethyl-2-oxo-1,3-oxazolidin-4-yl)methyl]phenoxy}benzylidene)-1,3-thiazolidine-2,4-dione;
22) 5-(3-fluoro-4-{4-[(3-ethyl-2-oxo-1,3-oxazolidin-4-yl)methyl]phenoxy}benzylidene)-1,3-thiazolidine-2,4-dione;
23) 5-(3-methoxy-4-{4-[(3-ethyl-2-oxo-1,3-oxazolidin-4-yl)methyl]phenoxy}benzylidene)-1,3-thiazolidine-2,4-dione;
24) 5-(3-triflouromethyl-4-{4-[(3-ethyl-2-oxo-1,3-oxazolidin-4-yl)methyl]phenoxy}benzylidene)-1,3-thiazolidine-2,4-dione;
25) 5-(2-chloro-4-{4-[(3-ethyl-2-oxo-1,3-oxazolidin-4-yl)methyl]phenoxy}benzylidene)-1,3-thiazolidine-2,4-dione;
26) 5-(2-fluoro-4-{4-[(3-ethyl-2-oxo-1,3-oxazolidin-4-yl)methyl]phenoxy}benzylidene)-1,3-thiazolidine-2,4-dione;
27) 5-(2-fluoro-4-{4-[(3-methyl-2-oxo-1,3-oxazolidin-4-yl)methyl]phenoxy}benzylidene)-1,3-thiazolidine-2,4-dione;
28) 3-(4-{3-fluoro-4-[(2-oxo-1,3-oxazolidin-5-l)methyl]phenoxy}benzylidene)-1,3-dihydro-2H-indol-2-one;
29) 5-(3-chloro-4-{4-[(2-oxo-1,3-oxazolidin-4-yl)methyl]phenoxy}benzylidene)-1,3-thiazolidine-2,4-dione;
30) 3-(4-{3-chloro-4-[(2-oxo-1,3-oxazolidin-5-yl)methyl]phenoxy}benzylidene)-1,3-dihydro-2H-indol-2-one;
31) 5-(3-fluoro-4-{4-[(2-oxo-13-oxazolidin-4-yl)methyl]phenoxy}benzylidene)-1,3-thiazolidine-2,4-dione;
32) [(4-oxo-5-(4-{4-[(3-methyl-2-oxo-1,3-oxazolidin-4-yl)methyl]phenoxy}benzylidene)-2-thioxo-1,3-thiazolidin-3-yl]acetic acid;
33) 4-(4-{4-[(4-oxo-2-thioxo-1,3-thiazolidin-5-ylidene)methyl]phenoxy}benzyl)-1,3-oxazolidin-2-one;
34) 3-methyl-4-(4-{4-[(4-oxo-2-thioxo-1,3-thiazolidin-5-ylidene)methyl]phenoxy}benzyl)-1,3-oxazolidin-2-one;
35) 3-ethyl-4-(4-{4-[(4-oxo-2-thioxo-1,3-thiazolidin-5-ylidene)methyl]phenoxy}benzyl)-1,3-oxazolidin-2-one;
36) [(4-oxo-5-(4-{4-[(2-oxo-1,3-oxazolidin-4-yl)methyl]phenoxy}benzylidene)-2-thioxo-1,3-thiazolidin-3-yl]acetic acid;
37) [(4-oxo-5-(4-{4-[(3-ethyl-2-oxo-1,3-oxazolidin-4-yl)methyl]phenoxy}benzylidene)-2-thioxo-1,3-thiazolidin-3-yl]acetic acid;
38) 4-(4-{2-fluoro-4-[(4-oxo-2-thioxo-1,3-thiazolidin-5-ylidene)methyl]phenoxy}benzyl)-3-methyl-1,3-oxazolidin-2-one;
39) [(4-oxo-5-(3-fluoro-4-{4-[(3-methyl -2-oxo-1,3-oxazolidin-4-yl)methyl]phenoxy}benzylidene)-2-thioxo-1,3-thiazolidin-3-yl]acetic acid;
40) 4-(4-{2-chloro-4-[(4-oxo-2-thioxo-1,3-thiazolidin-5-ylidene)methyl]phenoxy}benzyl)-3-methyl-1,3-oxazolidin-2-one;
41) [4-oxo-5-(3-chloro-4-{4-[(3-methyl -2-oxo-1,3-oxazolidin-4-yl)methyl]phenoxy}benzylidene)-2-thioxo-1,3-thiazolidin-3-yl]acetic acid;
42) 4-(4-{2-fluoro-4-[(4-oxo-2-thioxo-1,3-thiazolidin-5-yl)methyl]phenoxy}benzylidene)-3-ethyl-1,3-oxazolidin-2-one;
43) 4-(4-{2-methoxy-4-[(4-oxo-2-thioxo-1,3-thiazolidin-5-yl)methyl]phenoxy}benzylidene)-3-ethyl-1,3-oxazolidin-2-one;
44) 4-(4-{2-chloro-4-[(4-oxo-2-thioxo-1,3-thiazolidin-5-ylidene)methyl]phenoxy}benzyl)-3-ethyl-1,3-oxazolidin-2-one
45) 4-(4-{3-chloro-4-[(4-oxo-2-thioxo-1,3-thiazolidin-5-ylidene)methyl]phenoxy}benzyl)-3-ethyl-1,3-oxazolidin-2-one;
46) 3-ethyl-4-(4-{3-fluoro-4-[(4-oxo-2-thioxo-1,3-thiazolidin-5-ylidene)methyl]phenoxy}benzyl)-1,3-oxazolidin-2-one;
47) 4-(4-{2-methoxy-4-[(3-aceticacid-4-oxo-2-thioxo-1,3-thiazolidin-5-yl)ethyl]phenoxy}-benzyl)-3-methyl-1,3-oxazolidin-2-one;
48) 4-(4-{3-chloro-4-[(3-aceticacid-4-oxo-2-thioxo-1,3-thiazolidin-5-yl)ethyl]phenoxy}-benzyl)-3-methyl-1,3-oxazolidin-2-one;
49) 4-(4-{2-chloro-4-[(3-aceticacid-4-oxo-2-thioxo-1,3-thiazolidin-5-yl)ethyl]phenoxy}-benzyl)-3-ethyl-1,3-oxazolidin-2-one;
50) 4-(4-{2-methoxy-4-[(3-aceticacid-4-oxo-2-thioxo-1,3-thiazolidin-5-yl)ethyl]phenoxy}-benzyl)-3-ethyl-1,3-oxazolidin-2-one;
51) 4-(4-{2-triflouromethyl-51) 4-[(3-acetic acid-4-oxo-2-thioxo-1,3-thiazolidin-5-yl)ethyl]phenoxy}-benzyl)-3-ethyl-1,3-oxazolidin-2-one;
52) 5-(3-fluoro-4-{4-[(3-ethyl-2-oxo-1,3-oxazolidin-4-yl)methyl]phenoxy}benzylidene)-4-oxo-2-thioxo-1,3-thiazolidin-3-yl]acetic acid;
53) [4-oxo-5-(2-chloro-4-{4-[(3-ethyl-2-oxo-1,3-oxazolidin-4-yl)methyl]phenoxy}benzylidene)-2-thioxo-1,3-thiazolidin-3-yl]acetic acid;
54) [(4-oxo-5-(2-fluoro-4-{4-[(3-ethyl-2-oxo-1,3-oxazolidin-4-yl)methyl]phenoxy}benzylidene)-2-thioxo-1,3-thiazolidin-3-yl]acetic acid;
55) 4-(4-{2-triflouromethyl-4-[(4-oxo-2-thioxo-1,3-thiazolidin-5-yl)methyl]phenoxy}benzylidene)-3-methyl-1,3-oxazolidin-2-one;
56) [4-oxo-5-(3-trifluoromethyl-4-(4-[(3-methyl-2-oxo-1,3-oxazolidin-4-yl)methyl]phenoxy}benzylidene)-2-thioxo-1,3-thiazolidin-3-yl]acetic acid;
57) [(4-oxo-5-(2-fluoro-4-{4-[(3-methyl-2-oxo-1,3-oxazolidin-4-yl)methyl]phenoxy}benzylidene)-2-thioxo-1,3-thiazolidin-3-yl]acetic acid;
58) 4-(4-{3-chloro-4-[(4-oxo-2-thioxo-1,3-thiazolidin-5-ylidene)methyl]phenoxy}benzyl)-3-methyl-1,3-oxazolidin-2-one;
59) 4-(4-{2-triflouromethyl-4-[(4-oxo-2-thioxo-1,3-thiazolidin-5-yl)methyl]phenoxy}benzylidene)-3-ethyl-1,3-oxazolidin-2-one;
60) 4-(4-{2-chloro-4-[(4-oxo-2-thioxo-1,3-thiazolidin-5-ylidene)methyl]phenoxy}benzyl)-1,3-oxazolidin-2-one;
61) 5-(4-{4-[(3-methyl-2-oxo-1,3-oxazolidin-4-yl)methyl]phenoxy}benzyl)-1,3-thiazolidine-2,4-dione;
62) 5-(4-{4-[(3-ethyl-2-oxo-1,3-oxazolidin-4-yl)methyl]phenoxy)benzyl)-1,3-thiazolidine-2,4-dione;
63) 5-(3-fluoro-4-{4-[(3-methyl-2-oxo-1,3-oxazolidin-4-yl)methyl]phenoxy}benzyl)-1,3-thiazolidine-2,4-dione;
64) 5-(4-{4-[(3-methyl-2-oxo-1,3-oxazolidin-4-yl)methyl]phenoxy}benzyl)-1,3-dihydro-2H-indol-2-one;
65) 5(3-trifluoromethyl-(4-{4-[(3-ethyl-2-oxo-1,3-oxazolidin-4-yl)methyl]phenoxy}benzyl)-1,3-dihydro-2H-indol-2-one;
66) 5(3-fluoro-(4-{4-[(3-ethyl-2-oxo-1,3-oxazolidin-4-yl)methyl]phenoxy}benzyl)-1,3-dihydro-2H-indol-2-one;
67) 5(3-methoxy-(4-{4-[(3-methyl-2-oxo-1,3-oxazolidin-4-yl)methyl]phenoxy}benzyl)-1,3-dihydro-2H-indol-2-one
b 68) 5-(4-{4-[(3-ethyl-2-oxo-1,3-oxazolidin-4-yl)methyl]phenoxy}benzyl)-1,3-dihydro-2H-indol-2-one;
69) 5(3-methoxy-(4-{4-[(3-ethyl-2-oxo-1,3-oxazolidin-4-yl)methyl]phenoxy}benzyl)-1,3-dihydro-2H-indol-2-one;
70) 5-(2-chloro-4-{4-[(3-methyl-2-oxo-1,3-oxazolidin-4-yl)methyl]phenoxy}benzyl)-1,3-thiazolidine-2,4-dione;
71) 4-{4-[(3-methyl-2-oxo-1,3-oxazolidin-4-yl)methyl]phenoxy}-3-(trifluoromethyl benzyl]-1,3-thiazolidine-2,4-dione;
72) 3-chloro-4-{4-[(3-methyl-2-oxo-1,3-oxazolidin-4-yl)methyl]phenoxy}benzyl)-1,3-thiazolidine-2,4-dione;
73) 5-(3-chloro-4-{4-[(3-ethyl-2-oxo-1,3-oxazolidin-4-yl)methyl]phenoxy}benzyl)-1,3-thiazolidine-2,4-dione;
74) 5-(3-methoxy-4-{4-[(3-methyl-2-oxo-1,3-oxazolidin-4-yl)methyl]phenoxy}benzyl)-1,3-thiazolidine-2,4-dione;
75) 5-(3-trifluoromethyl-(4-{4-[(3-methyl-2-oxo-1,3-oxazolidin-4-yl)methyl]phenoxy}benzyl)-1,3-dihydro-2H-indol-2-one;
76) 5-(4-{4-[(3-methyl-2-oxo-1,3-oxazolidin-4-yl)methyl]phenoxy}benzyl)-2-thioxo-1,3-thiazolidin-3-yl]acetic acid;
77) 3-methyl-4-(4-{4-[(4-oxo-2-thioxo-1,3-thiazolidin-5-yl)methyl]phenoxy}benzyl)-1,3-oxazolidin-2-one;
78) 3-ethyl-4-(4-{4-[(4-oxo-2-thioxo-1,3-thiazolidin-5-yl)methyl]phenoxy}benzyl)-1,3-oxazolidin-2-one;
79) 5-(4-{4-[(3-ethyl-2-oxo-1,3-oxazolidin-4-yl)methyl]phenoxy}benzyl)-2-thioxo-1,3-thiazolidin-3-yl]acetic acid;
80) 4-(4-{2-fluoro-4-[(4-oxo-2-thioxo-1,3-thiazolidin-5-yl)methyl]phenoxy}benzyl)-3-methyl-1,3-oxazolidin-2-one;
81) 5-(3-fluoro-4-{4-[(3-methyl-2-oxo-1,3-oxazolidin-4-yl)methyl]phenoxy}benzyl)-4-oxo-2-thioxo-1,3-thiazolidin-3-yl]acetic acid;
82) 4-(4-{2-chloro-4-[(4-oxo-2-thioxo-1,3-thiazolidin-5-yl)methyl]phenoxy}benzyl)-3-methyl-1,3-oxazolidin-2-one;
83) 4-(4-{2-chloro-4-[(4-oxo-2-thioxo-1,3-thiazolidin-5-yl)methyl]phenoxy}benzyl)-3-ethyl-1,3-oxazolidin-2-one;
84) 4-(4-{2-fluoro-4-[(4-oxo-2-thioxo-1,3-thiazolidin-5-yl)methyl]phenoxy}benzyl)-N-ethyl-1,3-oxazolidin-2-one;
85) 4-{4-[4-[(4-oxo-2-thioxo-1,3-thiazolidin-5-yl)methyl]-2-(trifluoromethyl)phenoxy}benzyl)-N-ethyl-1,3-oxazolidin-2-one;
86) 5-(3-trifluoromethyl-4-{4-[(3-methyl-2-oxo-1,3-oxazolidin-4-yl)methyl]phenoxy}benzyl)-4-oxo-2-thioxo-1,3-thiazolidin-3-yl]acetic acid;
87) 5-(3-trifluoromethyl-4-{4-[(3-ethyl-2-oxo-1,3-oxazolidin-4-yl)methyl]phenoxy}benzyl)-4-oxo-2-thioxo-1,3-thiazolidin-3-yl]acetic acid;
88) 5-(2-chloro-4-{4-[(3-ethyl-2-oxo-1,3-oxazolidin-4-yl)methyl]phenoxy}benzyl)-4-oxo-2-thioxo-1,3-thiazolidin-3-yl]acetic acid;
89) 4-{4-[4-[(4-oxo-2-thioxo-1,3-thiazolidin-5-yl)methyl]-2-(trifluoromethyl)phenoxy]benzyl}-N-methyl-1,3-oxazolidin-2-one
90) 5-(3-chloro-4-{4-[(3-methyl-2-oxo-1,3-oxazolidin-4-yl)methyl]phenoxy}benzyl)-4-oxo-2-thioxo-1,3-thiazolidin-3-yl]acetic acid
91) 4-(4-{3-fluoro-4-[(4-oxo-2-thioxo-1,3-thiazolidin-5-yl)methyl]phenoxy}benzyl)-3-methyl-1,3-oxazolidin-2-one and
92) 4-(4-{3-chloro-4-[(4-oxo-2-thioxo-1,3-thiazolidin-5-yl)methyl]phenoxy}benzyl)-3-methyl-1,3-oxazolidin-2-one.

4. A pharmaceutical composition, which comprises a pharmaceutically effective amount of a Novel heterocyclic derivatives of formula (I) 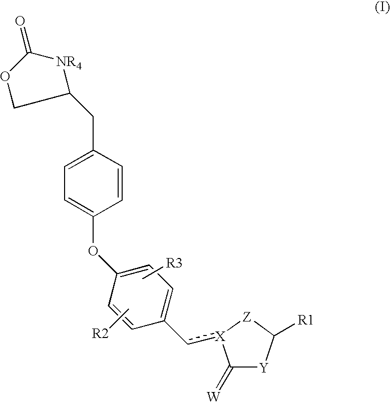
as defined in claim 1 and a pharmaceutically acceptable carrier, diluent, excipient or solvents.

5. A pharmaceutical composition as claimed in claim 4, in the form of a tablet, capsule, powder, syrup, solution, aerosol or suspension.

6. A method for reducing blood glucose, free fatty acids, cholesterol, triglycerides levels in plasma comprising administration an effective amount of a compound of formula (I) as defined in claim 1 to patient need thereof.

7. A method for treating obesity, autoimmune, inflammation, immunological, cancer disease comprising administration an effective amount of a compound of formula (I) as defined in claim 1 to patient need thereof.

8. A method for treating a disorder associated with insulin resistance comprising administrating as effective amount of a compound of formula (I) as defined in claim 1 to patient in need thereof.

9. The compound as claimed in claim 1, wherein said pharmaceutical acceptable salt is selected from hydrochloride, hydrobromide, sodium, potassium or magnesium salt.

10. A method for reducing blood glucose in plasma without adipogenic potential comprising administration an effective amount of a compound as claimed in claim for a compound as claimed in claim 3 to a mammal in need thereof.

11. A method for reducing TNF alfa, IL-6 and IL-beta comprising administration an effective amount of a compound as claimed in claim for a compound as claimed in claim 3 to a mammal in need thereof.

12. A method for reducing cancer cell progression comprising administration an effective amount of a compound as claimed in claim for a compound as claimed in claim 3 to a mammal in need thereof.
